(19) United States
(12) Patent Application Publication
(10) Pub. No.: US 2009/0185135 A1
(43) Pub. Date: Jul. 23, 2009

(54) Real image forming eye examination lens utilizing two reflecting surfaces providing upright image

(76) Inventor: Donald A. Volk, Honolulu, HI (US)

Correspondence Address: Donald A. Volk, 3872 Owena Street, Honolulu, HI, 96815, US

(21) Appl. No.: 12/321,709

(22) Filed: Jan. 22, 2009

Related U.S. Application Data
(60) Provisional application No. 61/062,004, filed on Jan. 22, 2008.

(51) Int. Cl. A61B 3/125; A61B 3/14

(52) U.S. Cl. 351/219; 351/206; 264/1.1; 264/1.9

(57) ABSTRACT

A diagnostic and therapeutic contact lens is provided for use with biomicroscopes for the examination and treatment of structures of the eye. The lens comprises a contacting surface adapted for placement on the cornea of an eye, two reflecting surfaces, and a refracting surface. A light ray emanating from the structure of the eye enters the lens and contributes to the formation of a correctly oriented real image. The light ray is reflected in an ordered sequence of reflections, first as a negative reflection in a posterior direction from an anterior reflecting surface and next as a positive reflection in an anterior direction from a posterior reflecting surface. The light ray contributes to forming the image of the structure of the eye either anterior to the lens or within the lens and proceeds along a pathway to the objective lens of the biomicroscope used for stereoscopic viewing and image scanning.

### PRIORITY CLAIM

This application claims priority to, and the full benefit of, U.S. Provisional Patent Application No. 61/062,004, titled “REAL IMAGE FORMING EYE EXAMINATION LENS UTILIZING TWO REFLECTING SURFACES PROVIDING UPRIGHT IMAGE” and filed Jan. 22, 2008, which is incorporated by reference in its entirety.

### BACKGROUND OF THE INVENTION

1. Field of the Invention

The lens of the present disclosure relates to ophthalmoscopic lenses for use with the slit lamp or other biomicroscope. More particularly the invention relates to diagnostic and therapeutic gonioscopic and indirect ophthalmoscopic contact lenses that incorporate two reflecting surfaces which combine to provide positive power contributing to the formation of a real and correctly oriented image of the examined structures of the eye anterior of the lens or within the lens or element of the lens while optimally directing the light rays to the objective lens of the slit lamp biomicroscope for stereoscopic viewing and image scanning.

2. Description of Prior Art

Eye examination lenses including indirect and direct ophthalmoscopy and gonioscopy lenses are used by ophthalmologists and optometrists for the diagnosis and treatment of the internal structures of the eye in conjunction with a slit lamp or other biomicroscope. Indirect ophthalmoscopy lenses, such as the Volk 90D lens, generally comprise a single lens with two refracting surfaces that combine to provide positive power contributing to the formation of a real image of the patient's eye fundus anterior of the examined eye. Direct ophthalmoscopy lenses, such as the Hruby lens, use minus power to produce a virtual image of the patient's eye fundus generally posterior of the examination lens. Some indirect and direct ophthalmoscopic lenses are pre-set or hand held in front of the patient's eye while others incorporate a contacting means and interface with the cornea and tear layer of the eye. An example of a contact indirect ophthalmoscopy lens would be the Volk QuadrAspheric® lens and an example of a contact direct ophthalmoscopy lens would be the Volk Centralis Direct® lens. Indirect ophthalmoscopy lenses provide a wide field inverted view while direct ophthalmoscopy lenses provide a small field with high magnification and high resolution in correct orientation.

Diagnostic lenses such as the Goldmann lens, Zeiss four mirror gonioscopy lens and Keoppe lens contact the eye and are used to examine and treat structures of the anterior chamber of the eye, specifically in the area of the anterior chamber angle, or iridocorneal angle. The four mirror lens incorporates angulated mirrors and like the other gonioscopy lenses operates to eliminate the power of the cornea to avoid total internal reflection of the light rays at the cornea-air interface. Light rays from the anterior chamber angle enter the lens and are reflected by mirrors along the line of vision of the viewer, one for each quadrant of the examined eye. In that a single mirror is used for each of the four sectional views, each image is reverted and discontinuous from the other sectional views. Furthermore the field of view obtainable through each mirror is very small. The Goldmann lens performs in an identical manner to the Zeiss four mirror lens except that it has only a single mirror used for gonioscopy. The Keoppe lens employs a contact lens having a rather highly curved convex anterior surface and a thickness sufficient to prevent total internal reflection of incident light rays from the anterior chamber angle from its convex surface, thereby allowing light rays to pass through for examination purposes. There is no real conjugate pupil formed by the Keoppe lens and the physician may only obtain a small field of view at an extremely angled inclination relative to the eye axis through a stereoscopic viewer.

Real image forming ‘indirect ophthalmoscopic’ viewing systems have also been suggested for viewing structures of the anterior chamber. An advantage of such a system lies in the continuous and uninterrupted 360 degree field of view that may be provided in the form of an annular section corresponding to the structures of the anterior chamber angle, viewed with the slit lamp biomicroscope in its normal orientation. Such a system is described in U.S. Pat. No. 6,164,779 to Volk. This patent sets forth a series of lenses comprising a first corneal contacting lens system receiving light rays originating at the anterior chamber angle and a second imaging forming system receiving light rays from the first lens system producing a real image of the anterior chamber angle outside of the patient's eye. Various embodiments include refracting as well as reflecting surfaces providing positive power for focusing light rays. Although the U.S. Pat. No. 6,164,779 patent presents the first real image forming gonioscopy lens system of its day, the complexity of a number of embodiments as well as an insufficiency of others to provide correction of chromatic and other aberrations prevented commercialization of this invention. U.S. Pat. No. 7,144,111 to Ross, III, et al., represents an attempt to provide an improved real image forming gonioscopy lens. Although achromatized and somewhat corrected for other aberrations, the lenses depicted in the embodiments of the 111 patent to Ross exhibit numerous disadvantages that preclude its successful application, including excessive weight, an excessive lens length of over 35 mm, an excessive distance from the examined eye to the image plane of over 51 mm, which is beyond the positioning range of the slit lamp biomicroscope, and poor stereoscopic visualization and image scanning capability resulting from the small light ray footprint at the biomicroscope objective lens aperture. In my co-pending patent application entitled ‘Real Image Forming Eye Examination Lens Utilizing Two Reflecting Surfaces’ I disclose an eye examination lens particularly well suited for gonioscopic examination of the eye. The lens provides a continuous and uninterrupted annular field of view of the anterior chamber angle as an inverted image viewed stereoscopically and having excellent optical quality.

### SUMMARY OF THE INVENTION

Based on the foregoing there is found to be a need to provide a real image forming gonioscopy lens that avoids the problems associated with the prior art lenses and which in particular provides a correctly oriented image of the structures of the eye, has excellent optical attributes, is easily positioned and manipulated within the orbital area of the examined eye and which avoids complexity of design and difficulty of manufacture. It is therefore a main object of the invention to provide an improved diagnostic and therapeutic gonioscopy lens that incorporates two reflecting surfaces that combine to provide positive power contributing to the formation of a real image that is correctly oriented with respect to the structures of the eye.

It is another object of the invention to provide a diagnostic and therapeutic gonioscopy lens that provides a continuous and uninterrupted annular field of view.

It is another object of the invention to provide a diagnostic and therapeutic gonioscopy lens that is well corrected for optical aberrations including field curvature, astigmatic error and chromatic aberration.

It is another object of the invention to provide a diagnostic and therapeutic gonioscopy lens that comprises as few as one or two optical elements.

It is another object of the invention to provide a diagnostic and therapeutic indirect ophthalmoscopy lens that incorporates two reflecting surfaces that combine to provide positive power contributing to the formation of a correctly oriented real image.

It is another object of the invention to provide a diagnostic and therapeutic indirect ophthalmoscopy lens that provides a continuous and uninterrupted annular field of view of the peripheral retina.

It is another object of the invention to provide a diagnostic and therapeutic indirect ophthalmoscopy lens that provides a sectional field of view of the retina.

These and other objects and advantages are accomplished by a diagnostic and therapeutic eye examination lens that incorporates two reflecting surfaces that work in concert to provide positive power contributing to the formation of a correctly oriented real image. The optical materials selected and curvatures provided result in a lens with improved optical quality, practicality of function and simplicity of design.

The lens of the present disclosure functions as both a condensing lens, directing light from the illumination portion of a biomicroscope to the visualized eye structures, and an image forming lens, producing a real image of the illuminated eye structures in an image plane anterior of the examined eye. The light pathways through the lens are folded across the lens axis through the use of two reflecting surfaces that optimally correct optical aberrations while shortening the distance to the plane of the real image.

The ophthalmoscopic contact lenses described in this disclosure may be used for general diagnosis as well as for treatment by means of the delivery of laser energy to the trabecular meshwork and adjacent iris structures of the eye, i.e., laser trabeculoplasty, peripheral laser iridoplasty, laser iridotomy, and in the delivery of laser energy in the treatment of the equatorial and peripheral retina. The term “ophthalmoscopic contact lens” as used in this disclosure refers to a contact lens for diagnosis or laser treatment of the interior structures of the eye including those of the fundus within the posterior chamber and the iris and iridocorneal angle within the anterior chamber.

In the lens of the present disclosure a light ray proceeding through the lens from the examined eye to the correctly oriented real image is reflected in an ordered sequence of reflections first in a first lens part as a negative reflection in a posterior direction from the anterior reflecting surface and next in a second lens part as a positive reflection in an anterior direction from the posterior reflecting surface.

A ‘negative reflection’ is defined as a reflected light ray that proceeds from the point of reflection closer to the axis of the lens than the incident ray as determined by the point of intersection of each with a perpendicular to the axis of the lens, the intersection occurring on one side of a plane that intersects the lens axis in a line.

Conversely, a ‘positive reflection’ is defined as a reflected light ray that proceeds from the point of reflection further from the axis of the lens than the incident ray as determined by the point of intersection of each with a perpendicular to the axis of the lens, the intersection occurring on one side of a plane that intersects the lens axis in a line.

The term ‘first reflected’ as used herein is descriptive of the first reflection in the ordered sequence of reflections. The term ‘next reflected’ is descriptive of the second reflection in the ordered sequence of reflections.

By ‘posterior direction’ is meant the direction of a reflected light ray towards the examined eye with reference to the Z axis, the Z axis being known to those skilled in the art as defining the coordinate dimension along or parallel to the axis of the lens. By ‘anterior direction’ is meant the direction of a reflected light ray away or further from the examined eye with reference to the Z axis. In the figures included in this disclosure the examined eye is shown on the left side or −Z position relative to the lens and the lens is shown on the right side or +Z position relative to the examined eye, therefore light rays reflected in a −Z direction relative to the point of reflection are reflected in a posterior direction and light rays reflected in a +Z direction relative to the point of reflection are reflected in an anterior direction.

The ‘first lens part’ is herein defined as a section of the lens in which the first reflection of a light ray occurs on one side of a plane that intersects the lens axis in a line. The ‘second lens part’ is herein defined as a section of the lens in which the second reflection of a light ray occurs on the opposite side of the plane defining the first lens part.

In some embodiments a single element consisting of two reflecting and refracting surfaces may comprise the entire lens. In other embodiments additional lens elements may be incorporated to enhance the optical qualities of the lens.

The lens may be produced of either a polymeric material such as polymethylmethacrylate (pmma), polycarbonate, polystyrene, ally diglycol carbonate (CR-39®) or other suitable polymeric material or a glass material, for example N-BK7 (available from Schott AG), S-LAH58 (available from Ohara Corporation) or any other optical glass types including glasses with refractive indices ranging from below Nd=1.5 to above Nd=1.9 or greater.

In the lens of the present disclosure the surface that comprises the anterior reflector and the refracting portion it surrounds may comprise a surface of continuous curvature, wherein both the reflecting and refracting portions are defined by the same surface parameters as a single curvature. Alternatively the surface may comprise a lenticular surface, wherein the reflecting portion and the refracting portion each are defined by different surface parameters as different curvatures. The anterior reflector surface may be spherical or aspheric, and if aspheric may comprise a polynomial-defined asphere. The refracting portion may be concave, plano or convex.

All refracting surfaces in the various embodiments disclosed other than the contacting surface adapted for placement on a cornea may be concave, convex, plano or defined as a polynomial surface having both concave and convex attributes, including the surface of a multi-element lens opposite the contacting surface in embodiments wherein a posterior refracting surface adjoins the posterior reflecting surface, thereby providing a contacting element that is bi-concave, plano-concave or meniscus in shape.

As an alternative to the use of optical cement as an interface medium in the various multiple element lens embodiments shown and described in this disclosure, gel and liquid interface mediums may be utilized instead, thus allowing separation of the component elements for sterilization purposes. A liquid or gel medium also provides a means to interface an intermediate or anterior glass reflecting element with a separate and disposable contacting portion comprising the contacting element and an open ended frustoconically shaped container for receiving the reflecting portion. The curvatures of two surfaces optically coupled at the interface of an optically coupled lens need not have exactly the same curvature and may have different curvatures.

The various embodiments shown are well corrected for chromatic aberration as the reflecting surfaces provide significant positive power contributing to the formation of the real image thus allowing the refracting surfaces to be tailored to minimize or practically eliminate dispersion.

Scanning of the real image may be accomplished by lateral and vertical movement of the biomicroscope and in conjunction with angulation or tilting of the gonioscopy lens on the eye the visualized area may be expanded to include a larger extent of the iris and the inner corneal surface adjacent the iridocorneal angle.

Other features and advantages of the invention will become apparent from the following description of the invention in conjunction with the accompanying drawings.

### BRIEF DESCRIPTION OF THE DRAWINGS

FIG. 1 shows a lens layout and ray tracing of a three-element gonioscopy lens according to a first embodiment of the invention

FIG. 2a shows a detailed view of the lens of FIG. 1.

FIG. 2b shows a more detailed view of the light ray pathways illustrated in FIG. 2a.

FIG. 2c shows another lens layout and ray tracing of a three-element gonioscopy lens according to the first embodiment of the invention.

FIG. 3 shows a lens layout and ray tracing of an optically coupled three-element gonioscopy lens according to a second embodiment of the invention.

FIG. 4 shows a lens layout and ray tracing of an optically coupled two-element gonioscopy lens according to a third embodiment of the invention.

FIG. 5a shows a lens layout and ray tracing of a single element gonioscopy lens according to a fourth embodiment of the invention.

FIG. 5b shows another lens layout and ray tracing of a single element gonioscopy lens according to the fourth embodiment of the invention.

FIG. 6 shows the lens layout and ray tracing of a two-element indirect ophthalmoscopy contact fundus lens according to a fifth embodiment of the invention.

FIG. 7 shows the lens layout and ray tracing of a single element indirect ophthalmoscopy contact fundus lens according to a sixth embodiment of the invention.

FIG. 8 shows the lens layout and ray tracing of a single element indirect ophthalmoscopy contact fundus lens providing a large sectional field of view according to an seventh embodiment of the invention

### DETAILED DESCRIPTION

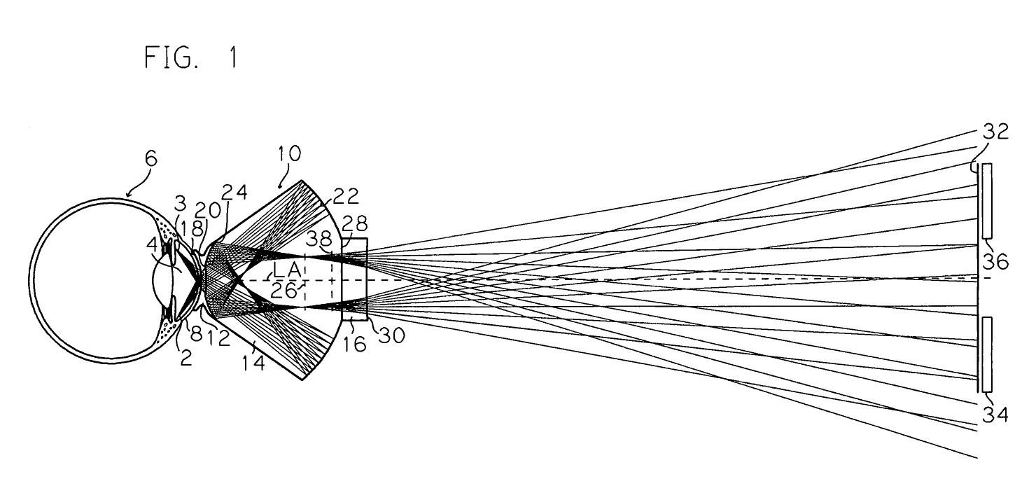

Referring to FIG. 1, there is shown a ray tracing and schematic cross-sectional view of an exemplary triplet gonioscopy lens according to a first embodiment of the invention, wherein lens 10 comprises an optically coupled lens including posterior contacting element 12, intermediate reflecting element 14 and anterior cap element 16. In this embodiment the anterior reflecting surface comprises an aspheric curvature and the posterior reflecting surface comprises a spherical curvature. Posterior element 12 is made of optical quality polymethylmethacrylate with an index of refraction of approximately Nd=1.492 and an Abbe number of approximately Vd=55.3, intermediate element 14 is made of S-LAH58 optical glass (available from Ohara Corp.) having an index of refraction of approximately Nd=1.883 and an Abbe number of approximately Vd=40.8, and anterior element 16 is made of N-BK7 (available from Schott AG) having an index of refraction of approximately Nd=1.517 and an Abbe number of approximately Vd=64. The three elements 12, 14 and 16 are optically coupled at their respective interfaces using a suitable optical coupling material, including one of a variety of adhesives known to those skilled in the art (such as NOA 68 or NOA 78 available from Norland Products or OP-24 Rev-B, the OP-4-20658 series, the OP4-20632 series, the OP-29V series and the OP-20 series of optical cements available from Dymax Corporation). In practice the lens is mounted in a holding frame or housing and applied to the cornea of a patient's eye in a manner similar to that used in conjunction with gonioscopic prisms and indirect ophthalmoscopic contact lens and which is generally known to those skilled in the art. For ease of illustration the frame is not included in the present or subsequent figures. As previously mentioned an optically clear liquid or gel (such as saline or ophthalmic methylcellulose) may be utilized instead of an optical cement as the optical interface medium. As used in this disclosure the term ‘optically coupled’ describes doublet or triplet lenses in which the lens elements are optically coupled or interfaced with a liquid, gel or cement interface material and the term ‘interface’ describes such an optically coupled interface. A liquid or gel optical coupling medium allows separation of the component elements for sterilization purposes or alternatively provides a means to interface an intermediate or anterior glass reflecting element with a separate and disposable contacting portion incorporating the contacting element. A cement interface provides means to optically couple lens elements in a fixed relationship not requiring additional support to maintain the relative positions of the lens elements, whereas a lens having lens elements optically coupled with a liquid or gel material requires a means to maintain relative position and alignment between the coupled elements. Such a means to maintain relative position and lens element alignment may include a housing or holding frame as above mentioned formed as a frustoconically shaped container portion comprising the contacting element at its small end and an opening at the opposite larger end for receiving the anterior reflecting element. A small measured amount of saline, methylcellulose or other suitable liquid or gel optical interface material may be placed in the container portion on the surface of the contacting element opposite the contacting surface prior to the insertion of the anterior element. Once the anterior element is inserted into the container portion and brought into contact with the liquid or gel material, the liquid or gel material will be made to conform to both interface surfaces it contacts, and to form a thin section as it seeps between the surfaces. An optical cement, or liquid or gel interface coupling medium used in conjunction with an appropriately designed housing as described, may be utilized in the present and subsequent exemplary lenses and lens embodiments where an optical interface is indicated.

For illustrative purposes, only two ray bundles are shown emanating from point sources on opposite sides of the axis of the lens within the anterior chamber of the schematic eye. Light ray bundle 2 emanates from an iridocorneal point source and light ray bundle 3 emanates from a peripheral iris point source. For ease of illustration, the tear film of the eye is not shown in the present or subsequent figures. Referring to FIG. 1, light rays of ray bundles 2 and 3 emanating from the stated iridocorneal and peripheral iris locations of anterior chamber 4 of eye 6 pass through the cornea 8 and tear layer of the eye and enter posterior contacting element 12 of lens 10 through corneal contacting surface 18 and continue through interface 20 into intermediate reflecting element 14 and to concave reflecting surface 22 from which each light ray is first reflected as a negative reflection in a posterior direction towards the axis of the lens, the light rays there forming an intermediate image (not identified) within the lens. Each light ray proceeds in its respective direction across lens axis LA and continues to concave reflecting surface 24 from which it is next reflected as a positive reflection in an anterior direction, contributing to the formation of final and correctly oriented real image 26. The light rays proceed from the real image in their respective directions towards interface 28 and enter anterior cap element 16 and continue to surface 30 where they are refracted and exit the lens. The rays proceed towards biomicroscope objective lens aperture 32 and enter left and right microscope lenses 34 and 36, respectively, of the observing stereomicroscope. The stereomicroscope is adjusted to focus at virtual image 38 to provide an upright and correctly oriented view of the observed structures of the eye.

As can be seen in FIG. 1 the ray span of both light bundles 2 and 3 at the plane of biomicroscope aperture 32 exceeds the extent of the biomicroscope aperture and the left and right microscope lenses 34 and 36, thus insuring binocular and stereoscopic biomicroscope visualization of the observed image both when the biomicroscope is coaxial with the lens as shown and when the biomicroscope is moved off axis to bring peripheral image points to a more central location of the visual field of the biomicroscope. The ray spans of lenses depicted in subsequent figures and embodiments likewise exceed the extent of the biomicroscope aperture and the left and right microscope lenses.

As an alternative to the standard slit lamp biomicroscope a CCD, CMOS or other sensor based camera system incorporating the lens may be focused at the plane of the virtual image, thus allowing the light rays of the formed image that are refocused on the CCD or CMOS sensor to be converted to an analog or digital signal and then converted to an image, series of images or continuous video sequence displayed on a video monitor in real time for immediate diagnostic applications or digitally stored for subsequent review, electronic transmission or other applications. A similar alternative application provides that a CCD, CMOS or other image sensor be placed at the image plane of the lens slightly modified in design and truncated at the anterior end, thus allowing the light rays of the formed image that are directly focused on the sensor in like manner to be converted to an analog or digital signal and converted to an image, series of images or continuous video sequence displayed on a video monitor in real time for immediate diagnostic applications or digitally stored for subsequent review, electronic transmission or other applications. Both of the above electronic imaging systems may be utilized in conjunction with the lens of the present disclosure including that of the present embodiment as well as those of subsequent embodiments.

Illumination of the anterior chamber structures may be provided by the slit lamp biomicroscope's illumination system in a typical manner. The par focal illumination system will provide light to the anterior chamber following similar light ray pathways as shown, from the image plane back through the lens and cornea to the anterior chamber. Alternatively, illumination may be provided through optical fibers or through the use of LED or OLED lamps positioned around lens element 16 the emitted light of which is converged and directed to pass through interface 20, cornea 8 and to the iris and iridocorneal angle, following similar but oppositely directed pathways to the rays emanating from the anterior chamber structures and proceeding to the first mirror surface, thereby illuminating selectively a portion of the anterior chamber or the entire circumference of the anterior chamber. Alternatively the optical fibers or LED's may direct their illumination along the outside of frustoconically shaped intermediate element 14 to or through contacting element 12 or directly to the cornea 8 of eye 6, thereby providing illumination of the anterior chamber without passing the illumination light rays through the lens. The above described fiber optic and LED illumination systems may be affixed to or detachably removable from the ophthalmoscopic contact lens and may be utilized in conjunction with the lens of the present embodiment as well as those of subsequent embodiments.

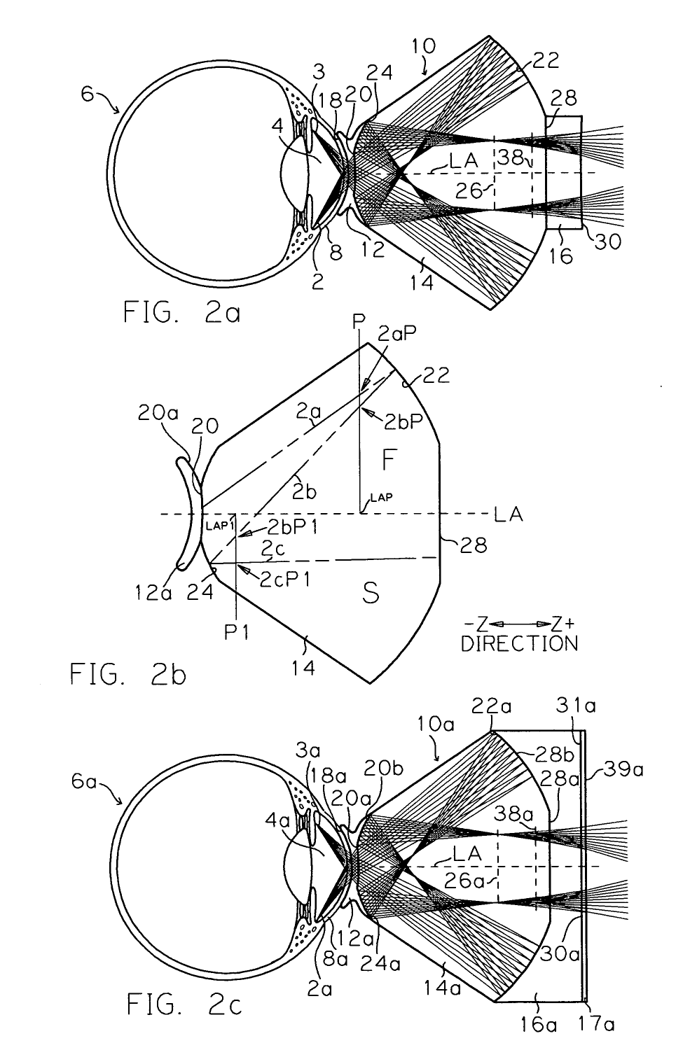

FIG. 2a shows the same lens as in FIG. 1 minus the diverging light rays proceeding from the lens to the plane of the biomicroscope in order to better illustrate the light ray pathways and lens elements and surfaces. As previously described, light rays of ray bundles 2 and 3 emanating from the stated iridocorneal and peripheral iris locations of anterior chamber 4 of eye 6 pass through the cornea 8 and tear layer of the eye and enter posterior contacting element 12 of lens 10 through corneal contacting surface 18 and continue through interface 20, comprised of the anterior and posterior surfaces of lens elements 12 and 14 respectively, optically coupled with an interface material. As the light rays enter intermediate reflecting element 14 they are bent towards the axis of the lens due to the high refractive index of the glass comprising element 14, thereby reducing the diameter required of concave reflecting surface 22 from which each light ray is first reflected as a negative reflection in a posterior direction towards the axis of the lens, the light rays there forming an intermediate image within the lens. Each light ray proceeds in its respective direction across lens axis LA and continues to concave reflecting surface 24 from which it is next reflected as a positive reflection in an anterior direction, contributing to the formation of final and correctly oriented real image 26. The light rays proceed from the real image in their respective directions towards interface 28, comprised of the anterior and posterior surfaces of lens elements 14 and 16 respectively, optically coupled with an interface material, and enter anterior cap element 16 and continue to surface 30 where they are refracted and exit the lens.

Contacting surface 18 comprises a concave surface adapted for placement on the patient's cornea, and may have a spherical or aspherical curvature. In the exemplary lens of this embodiment surface 18 has an apical radius of 7.7 mm and is aspheric. Optical interface 20 is the interface of the central refracting portions of the anterior and posterior surfaces respectively of lens elements 12 and 14. The curvature of interface 20 with respect to lens element 14 is spherical and concave with a radius of 20 mm. Optical interface 28 is the interface of the central refracting portions of the anterior and posterior surfaces respectively of lens elements 14 and 16. The curvature of interface 28 with respect to lens element 14 is piano. The optical coupling material used to optically couple the interface surfaces may be used advantageously to fill gaps, variable distances or mismatches between the two interface curvatures. As previously stated, the curvatures of two surfaces optically coupled at the interface of an optically coupled lens need not have exactly the same curvature and may have different curvatures. Referring to the figure, reflecting surface 22 has an aspheric concave curvature with an apical radius of 18.4 mm, and in combination with the plano curvature of optical interface 28 comprises a lenticulated surface as the anterior surface of lens element 14. By ‘lenticulated surface’ and ‘lenticulated design’ is meant a surface or surface design having discontinuous curvatures. Reflecting surface 22 provides plus power, converging light rays directed to it from concave corneal contacting surface 18. Reflecting surface 22 comprises an internally reflecting mirror-coated annular section having a 13 mm inner diameter that surrounds optical interface 28. Reflecting surface 24 has a spherical curvature with a radius of 10.88 mm, and in combination with the concave curvature of optical interface 20 comprises a lenticulated surface as the posterior surface of lens element 14. Reflecting surface 24 provides plus power, converging light rays directed to it from reflecting surface 22. Reflecting surface 24 also comprises an internally reflecting mirror-coated annular section having a 4.3 mm inner diameter that surrounds optical interface 20. The reflective sections may be mirrored by means of vacuum deposition of an evaporated or sputtered metal such as aluminum or silver, and protectively overcoated with a hardcoating, polymer or paint layer, as is known to those skilled in the art. Surface 30 of lens element 16 has a concave curvature with a radius of 90 mm. Anterior cap element 16 is approximately 4 mm thick and serves to unify and precisely position the left and right eye images comprising the stereoscopic view across the extent of the visualized field and to position virtual image 38 internally within the lens over 5 mm from concave refracting surface 30 of anterior cap element 16.

The exemplary lens as shown and described with reference to FIG. 2a, comprising a first anterior plus powered aspheric reflector paired with a second posterior plus powered spherical reflector, each which respectively produce the stated posterior and negative and anterior and positive reflections, provides a three element optical system for a diagnostic and therapeutic gonioscopy lens with excellent imaging qualities utilizing lenticulated designs for both the anterior and posterior surfaces of intermediate reflecting element 14.

The formula:

$$z = \frac{{cr}^{2}}{1 + \sqrt{1 - ( 1 + k ) {r}^{2} {r}^{2}}} + {a}_{1} r + {a}_{2} {r}^{2} {a}_{3} {r}^{3} … {a}_{n} {r}^{n}$$

has been utilized in defining the aspheric surfaces of this invention, where z equals the surface sag along the lens axis, c equals the curvature (i.e., reciprocal of the radius), r is the radial coordinate in lens units, k equals the conic constant, and aₙ (where n=1, 2, . . . ) is the coefficient value of any of the selected conic deformation terms.

Referring again to FIG. 2a, it may be noted that the diameter of the posterior end of contacting element 12 exceeds that of interface 20 thus allowing contacting element 12 to be advantageously shaped to function as an eyelid flange. An eyelid flange facilitates a positive interface with the tear or fluid layer of the eye when the patient tends to blink or squeeze the eyelids closed during the diagnostic or treatment procedure, and the use of such a flange is known to those skilled in the art. The anterior end of contacting element 12 extends beyond interface 20 and is as large in diameter as reflecting surface 24 to which it is interfaced, thus it provides protection to the mirror coating applied to surface 24.

FIG. 2b shows an alternate contacting element design 12a in which the anterior surface of element 12a has an annular convex portion 20a surrounding interface 20 thereby providing a large relief area for the patient's eye lids between surface 20a and mirror surface 24. The contact elements of subsequent figures and embodiments likewise may incorporate diameters, curvatures or recesses similar to that shown in FIG. 2a and FIG. 2b in order to provide a lid flange function and mirror protection as described.

As previously mentioned, in the lens of the present disclosure light rays proceeding through the lens from the examined eye to the real image are each reflected in an ordered sequence of reflections with the first reflection occurring from the anterior reflecting surface as a negative reflection in a posterior direction and with the second reflection occurring from the posterior reflecting surface as a positive reflection in an anterior direction. Also as previously mentioned, the first reflection of each light ray occurs in a first lens part and the second reflection occurs in a second lens part.

FIG. 2b shows an enlargement of intermediate reflecting element 14 and the pathway of one of the central rays of light ray bundle 2 shown in FIG. 2a, proceeding through the lens from interface 20 to interface 28, clearly illustrating how the reflections of individual rays conform first to the prescription of negative reflection from the first reflecting surface in a first lens part and second to the prescription of positive reflection from the second reflecting surface in a second lens part as described. Line P is perpendicular to lens axis LA and extends from the lens axis into first lens part F. LAP represents the point of intersection of line P and lens axis LA. Individual reflected light ray 2b proceeds from the portion of anterior reflecting surface 22 within first lens part F closer to lens axis LA than preceding incident ray 2a as demonstrated by each ray's respective intersection point 2bP and 2aP with line P and specifically as demonstrated by the lesser distance from 2bP to LAP compared to the greater distance from 2aP to LAP. Line P1 is perpendicular to lens axis LA and extends from the lens axis into second lens part S. LAP1 represents the point of intersection of line P1 and lens axis LA. Individual reflected light ray 2c proceeds from the portion of posterior reflecting surface 24 within second lens part S further from lens axis LA than preceding incident ray 2b as demonstrated by each ray's respective intersection point 2cP1 and 2bP1 with line P1 and specifically as demonstrated by the greater distance from 2cP1 to LAP1 compared to the lesser distance from 2bP1 to LAP1.

Light rays emanating from the area of the iridocorneal angle and peripheral iris and contributing to the formation of an upright and correctly oriented real image each reflect in this ordered sequence of reflections in the present as well as in subsequent embodiments and examples directed to anterior chamber examination and treatment lenses. Furthermore, light rays emanating from the fundus of the eye and contributing to the formation of an upright and correctly oriented real image each reflect in this ordered sequence of reflections in subsequent embodiments and examples directed to posterior chamber examination and treatment lenses. Any perpendicular line P or P1 extending from the lens axis into the first and second lens parts that intersects pairs of incident and reflected rays will demonstrate this property.

Referring to FIG. 2c, there is shown a ray tracing and schematic cross-sectional view of a second exemplary triplet gonioscopy lens according to the first embodiment of the invention, wherein lens 10a comprises an optically coupled lens including posterior contacting element 12a, intermediate element 14a and anterior cap element 16a. The lens also includes optically coupled plano cover glass element 17a. In this embodiment the anterior reflecting surface comprises an aspheric curvature incorporated into the posterior surface of anterior cap element 16a and the posterior reflecting surface comprises a spherical curvature incorporated into the anterior surface of contacting element 12a. Posterior element 12a is made of optical quality polymethylmethacrylate, intermediate element 14a is made of S-LAH58 optical glass, anterior element 16a is made of polymethylmethacrylate and cover glass 17a is made of N-BK7. The four elements 12a, 14a, 16a and 17a are optically coupled at their respective interfaces using suitable coupling materials as previously described. Referring to FIG. 2b, light rays of ray bundles 2a and 3a emanating from the stated iridocorneal and peripheral iris locations of anterior chamber 4a of eye 6a pass through the cornea 8a and tear layer of the eye and enter posterior contacting element 12a of lens 10a through corneal contacting surface 18a and continue through interface section 20a, comprised of the anterior and posterior surfaces of lens elements 12a and 14a respectively, optically coupled with an interface material. As the light rays enter intermediate reflecting element 14a they are bent towards the axis of the lens due to the high refractive index of the glass comprising element 14a, thereby reducing the diameter required of first reflecting surface. The light rays proceed through the convex anterior surface of intermediate lens element 14a and the adjacent annular section of interface 28b, comprised of the anterior and posterior surfaces of lens elements 14a and 16a respectively, optically coupled with an interface material and continue to concave reflecting surface 22a of lens element 16a from which each light ray is first reflected as a negative reflection in a posterior direction. The rays continue through the optical interface and convex anterior surface of intermediate lens element 14a towards the axis of the lens, the light rays there forming an intermediate image within the lens. Each light ray proceeds in its respective direction across lens axis LA and through the convex posterior surface of intermediate lens element 14a and the adjacent annular section of interface 20b, comprised of the anterior and posterior surfaces of lens elements 12a and 14a respectively, optically coupled with an interface material, and continues to concave reflecting surface 24a of lens element 12a from which it is next reflected as a positive reflection in an anterior direction. The light rays continue through the interface and convex posterior surface of intermediate lens element 14a, forming final and correctly oriented real image 26a. The light rays proceed from the real image in their respective directions towards the central plano section of interface section 28a, enter anterior cap element 16a, continue to surface 30a, proceed through interface 31a, enter cover glass element 17a and refract through surface 39a to exit the lens.

Contacting surface 18a comprises a concave surface adapted for placement on the patient's cornea and has an apical radius of 7.7 mm and is aspheric. The central 4.3 mm diameter section of interface 20a with respect to the interface curvature of lens element 14a is spherical and concave with a radius of 20 mm and the surrounding annular section of interface 20b with respect to the interface curvature of lens element 14a is spherical and convex with a radius of 10.88 mm. The central 13 mm diameter section of interface 28a with respect to the interface curvature of lens element 14a is plano and the surrounding annular section of interface 28b with respect to the interface curvature of lens element 14a is spherical and convex with a radius of 18.7 mm. Reflecting surface 22a has an aspheric concave curvature with an apical radius of 18.35 mm and provides plus power, converging light rays directed to it from concave corneal contacting surface 18a. Reflecting surface 22a comprises an externally reflecting mirror-coated annular section having a 13 mm inner diameter. Reflecting surface 24a has an aspheric concave curvature with an apical radius of 10.88 mm and provides plus power, converging light rays directed to it from reflecting surface 22a. Reflecting surface 24a also comprises an externally reflecting mirror-coated annular section and has a 4.3 mm inner diameter. The reflective sections may be mirrored by means of vacuum deposition as previously outlined and are encapsulated and protected within their respective interfaces. Surface 30a of lens element 16a is plano, anterior cap element 16a has a center thickness of approximately 5.0 mm and plano cover glass element 17a is approximately 1.25 mm thick. Virtual image 38a is positioned internally within the lens over 7.0 mm from refracting surface 30a of anterior cap element 16a.

The exemplary lens as shown and described with reference to FIG. 2c, comprising a first anterior plus powered aspheric reflector paired with a second posterior plus powered aspheric reflector, each which respectively produce the stated posterior and negative and anterior and positive reflections, provides a three element optical system, including an additional protective fourth element as a cover glass, for a diagnostic and therapeutic gonioscopy lens with excellent imaging qualities utilizing an intermediate glass element 14a comprising all spherical surfaces which may be easily and inexpensively manufactured and a polymeric anterior cap element 16a that may also be easily and accurately produced by a cast or injection molding.

Lenses of subsequent multi-element optically coupled lens embodiments may in like manner be designed with externally reflecting concave surfaces instead of internally reflecting concave surfaces with respect to either or both the first anterior reflecting surface and the second posterior reflecting surface. Furthermore, triplet designs comprising externally reflecting concave surfaces with respect to both the first anterior reflecting surface and the second posterior reflecting surface may incorporate a liquid or gel medium as the component material of the intermediate element rather than a solid plastic or glass material. For example, an optically clear mineral oil having a refractive index Nd=1.48 may be used as the optical medium between the anterior and posterior reflecting surfaces. A frustoconically shaped housing incorporating the contacting element and posterior reflector may be filled will the liquid medium and then hermetically sealed with a cap incorporating both the anterior reflector and central refracting portion.

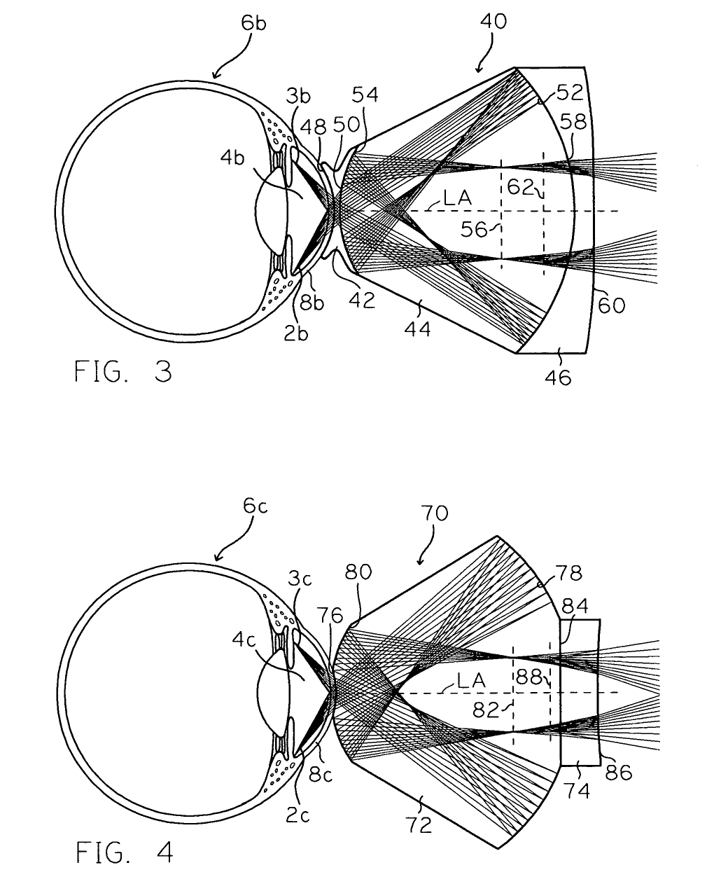

Referring to FIG. 3, there is shown a ray tracing and schematic cross-sectional view of an exemplary triplet gonioscopy lens according to a second embodiment of the invention, wherein lens 40 comprises an optically coupled lens including posterior contacting element 42, intermediate reflecting element 44 and anterior cap element 46. In this embodiment both the anterior and posterior reflecting surfaces comprise aspheric curvatures and both are non-lenticulated surfaces. Posterior element 42 is made of optical quality polymethylmethacrylate, intermediate element 44 is made of S-LAH58 optical glass and anterior element 46 is made of N-BK7. The three elements 42, 44 and 46 are optically coupled at their respective interfaces using suitable coupling materials as previously described. Referring to FIG. 3, light rays of ray bundles 2b and 3b emanating from the stated iridocorneal and peripheral iris locations of anterior chamber 4b of eye 6b pass through the cornea 8b and tear layer of the eye and enter posterior contacting element 42 of lens 40 through corneal contacting surface 48 and continue through interface 50, comprised of the anterior and posterior surfaces of lens elements 42 and 44 respectively, optically coupled with an interface material. As the light rays enter intermediate reflecting element 44 they are bent towards the axis of the lens due to the high refractive index of the glass comprising element 44, thereby reducing the diameter required of concave reflecting surface 52 from which each light ray is first reflected as a negative reflection in a posterior direction towards the axis of the lens, the light rays there forming an intermediate image within the lens. Each light ray proceeds in its respective direction across lens axis LA and continues to concave reflecting surface 54 from which it is next reflected as a positive reflection in an anterior direction, contributing to the formation of final and correctly oriented real image 56. The light rays proceed from the real image in their respective directions towards interface 58, comprised of the anterior and posterior surfaces of lens elements 44 and 46 respectively, optically coupled with an interface material, and enter anterior cap element 46 and continue to surface 60 where they are refracted and exit the lens.

Contacting surface 48 comprises a concave surface adapted for placement on the patient's cornea and has an apical radius of 7.7 mm and is aspheric. Optical interface 50 is the interface of the central refracting portions of the anterior and posterior surfaces respectively of lens elements 42 and 44. The curvature of interface 50 with respect to lens element 44 is aspheric and convex with an apical radius of 10.88 mm. Optical interface 58 is the interface of the central refracting portions of the anterior and posterior surfaces respectively of lens elements 44 and 46. The curvature of interface 58 with respect to lens element 44 is aspheric and convex with an apical radius of 18.7 mm. Reflecting surface 52 is a continuation of the curvature comprising interface 58 and in combination with the curvature of interface 58 forms a continuous curvature as the anterior surface of lens element 44. Reflecting surface 52 provides plus power, converging light rays directed to it from concave corneal contacting surface 48. Reflecting surface 52 comprises an internally reflecting mirror-coated annular section having a 13 mm inner diameter that surrounds optical interface 58. Reflecting surface 54 is a continuation of the curvature comprising interface 50 and in combination with the curvature of interface 50 forms a continuous curvature as the anterior surface of lens element 42. Reflecting surface 54 provides plus power, converging light rays directed to it from reflecting surface 52. Reflecting surface 54 also comprises an internally reflecting mirror-coated annular section having a 5.4 mm inner diameter that surrounds optical interface 50. The reflective sections may be mirrored by means of vacuum deposition and protectively overcoated as previously described. Surface 60 of lens element 46 has a plano curvature in its central refracting area. Virtual image 62 is positioned internally within the lens over 4.5 mm from refracting surface 60 of anterior cap element 46.

The exemplary lens as shown and described with reference to FIG. 3, comprising a first anterior plus powered aspheric reflector paired with a second posterior plus powered aspheric reflector, each which respectively produce the stated posterior and negative and anterior and positive reflections, provides a three element optical system for a diagnostic and therapeutic gonioscopy lens with excellent imaging qualities utilizing continuous surface curvatures for both the anterior and posterior surfaces of intermediate reflecting element 44.

Referring to FIG. 4, there is shown a ray tracing and schematic cross-sectional view of an exemplary doublet gonioscopy lens according to a third embodiment of the invention, wherein lens 70 comprises an optically coupled lens including posterior contacting and reflecting element 72 and anterior cap element 74. In this embodiment the anterior reflecting surface comprises an aspheric curvature and the posterior reflecting surface comprises a spherical curvature. Posterior element 72 is made of S-LAH58 optical glass and anterior element 74 is made of N-BK7. The two elements 72 and 74 are optically coupled at their interface using a suitable coupling material as previously described. Referring to FIG. 4, light rays of ray bundles 2c and 3c emanating from the stated iridocorneal and peripheral iris locations of anterior chamber 4c of eye 6c pass through the cornea 8c and tear layer of the eye and enter posterior contacting element 72 of lens 70 through corneal contacting surface 76 and continue to concave reflecting surface 78 from which each light ray is first reflected as a negative reflection in a posterior direction towards the axis of the lens, the light rays there forming an intermediate image within the lens. Each light ray proceeds in its respective direction across lens axis LA and continues to concave reflecting surface 80 from which it is next reflected as a positive reflection in an anterior direction, contributing to the formation of final and correctly oriented real image 82. The light rays proceed from the real image in their respective directions towards interface 84, comprised of the anterior and posterior surfaces of lens elements 72 and 74 respectively, optically coupled with an interface material, and enter anterior cap element 74 and continue to surface 86 where they are refracted and exit the lens.

Contacting surface 76 comprises a concave surface adapted for placement on the patient's cornea and has radius of 8.0 mm and is spherical. Optical interface 84 is the interface of the central refracting portions of the anterior and posterior surfaces respectively of lens elements 72 and 74. The curvature of interface 84 with respect to lens element 72 is plano. Reflecting surface 78 has an aspheric concave curvature with an apical radius of 18.4 mm and in combination with the plano curvature of optical interface 84 comprises a lenticulated surface as the anterior surface of lens element 72. Reflecting surface 78 provides plus power, converging light rays directed to it from concave corneal contacting surface 76. Reflecting surface 78 comprises an internally reflecting mirror-coated annular section having a 13 mm inner diameter that surrounds optical interface 84. Reflecting surface 80 has a spherical curvature with a radius of 10.88 mm, and in combination with the concave curvature of contacting surface 76 comprises a lenticulated surface as the posterior surface of lens element 72. Reflecting surface 80 provides plus power, converging light rays directed to it from reflecting surface 78. Reflecting surface 80 also comprises an internally reflecting mirror-coated annular section having a 4.3 mm inner diameter that surrounds concave contacting surface 76. The reflective sections may be mirrored by means of vacuum deposition and protectively overcoated as previously described. Surface 86 of lens element 74 has a concave curvature with a radius of 90 mm. Virtual image 88 is positioned internally within the lens over 4.5 mm from concave refracting surface 86 of anterior cap element 74.

The exemplary lens as shown and described with reference to FIG. 4, comprising a first anterior plus powered aspheric reflector paired with a second posterior plus powered spherical reflector, each which respectively produce the stated posterior and negative and anterior and positive reflections, provides a simplified two element optical system for a diagnostic and therapeutic gonioscopy lens with excellent imaging qualities utilizing lenticulated designs for both the anterior and posterior surfaces of contacting and reflecting element 72.

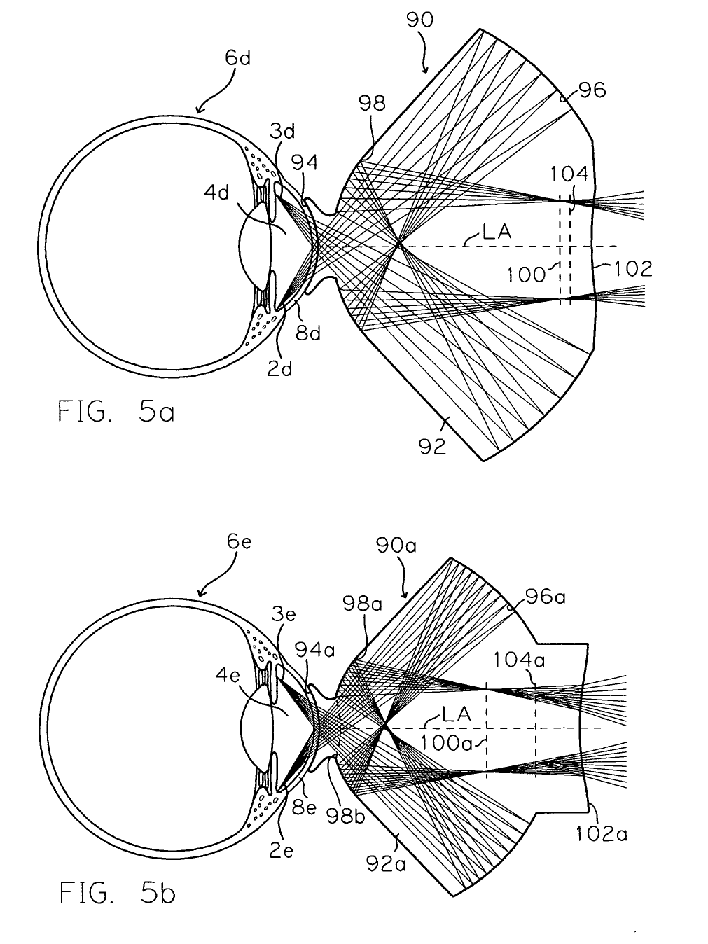

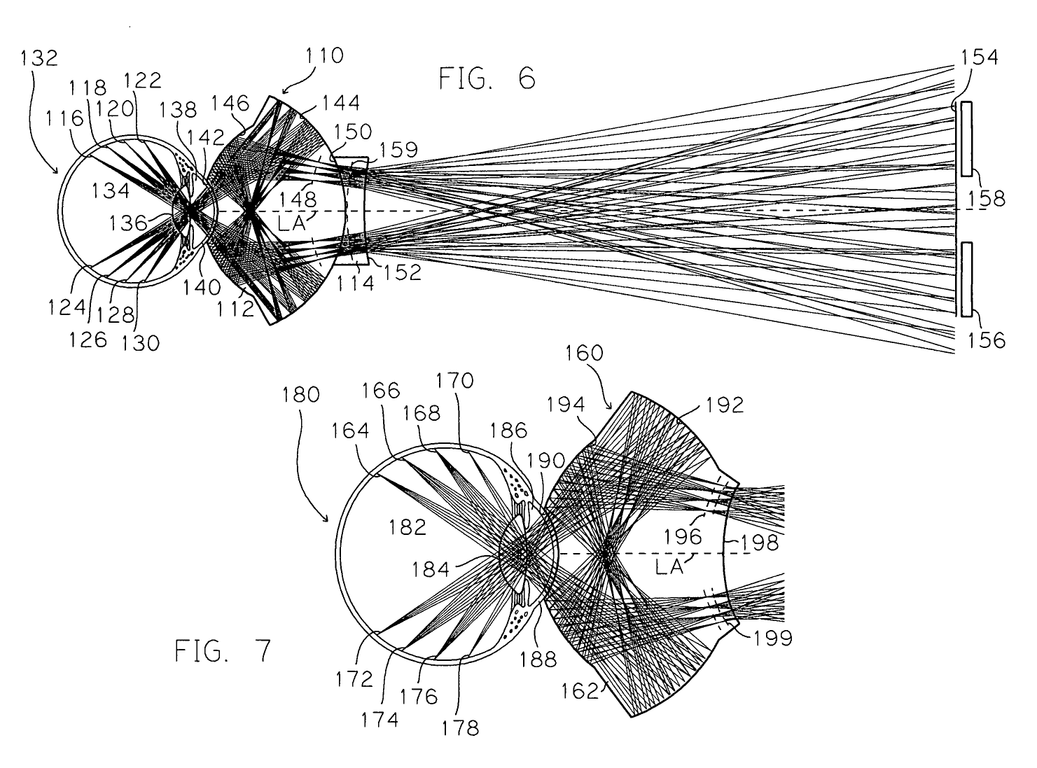

Referring to FIG. 5a, there is shown a ray tracing and schematic cross-sectional view of a lens layout of an exemplary single element gonioscopy lens 90 according to a fourth embodiment of the invention. In this embodiment both the anterior and posterior reflecting surfaces comprise aspheric curvatures, the posterior and anterior lens surfaces are lenticulated, and the posterior reflecting surface is displaced in an anterior direction from the contacting surface thereby providing a relief area for the patient's eyelids. The lens is made of optical quality polymethylmethacrylate. Referring to FIG. 5a, light rays of ray bundles 2d and 3d emanating from the stated iridocorneal and peripheral iris locations of anterior chamber 4d of eye 6d pass through the cornea 8d and tear layer of the eye and enter posterior contacting surface 94 of lens element 92 and continue to concave reflecting surface 96 from which each light ray is first reflected as a negative reflection in a posterior direction towards the axis of the lens, the light rays there forming an intermediate image within the lens. Each light ray proceeds in its respective direction across lens axis LA and continues to concave reflecting surface 98 from which it is next reflected as a positive reflection in an anterior direction, contributing to the formation of final and correctly oriented real image 100. The light rays proceed from the real image in their respective directions towards surface 102 through which they are refracted and exit the lens.

Contacting surface 94 comprises a concave surface adapted for placement on the patient's cornea and has an apical radius of 7.7 mm and is aspheric. Reflecting surface 96 has an aspheric concave curvature with an apical radius of 21.45 mm, and in combination with the concave curvature of surface 102 comprises a lenticulated surface as the anterior surface of the lens. Reflecting surface 96 provides plus power, converging light rays directed to it from concave corneal contacting surface 94. Reflecting surface 96 comprises an internally reflecting mirror-coated annular section having a 20 mm inner diameter that surrounds refracting surface 102. Reflecting surface 98 has an aspheric curvature with an apical radius of 13.31 mm, and in combination with the displaced concave curvature of contacting surface 94 comprises a lenticulated surface at the posterior end of the lens. Reflecting surface 98 provides plus power, converging light rays directed to it from reflecting surface 96. Reflecting surface 98 also comprises an internally reflecting mirror-coated annular section having a 6.2 mm inner diameter that surrounds the stemmed portion displacing it from concave contacting surface 94. The reflective sections may be mirrored by means of vacuum deposition and protectively overcoated as previously described. Surface 102 has a polynomial defined aspheric curvature with both concave and convex attributes. Virtual image 104 is positioned internally within the lens approximately 2 mm from refracting surface 102.

The exemplary lens as shown and described with reference to FIG. 5a, comprising a first anterior plus powered aspheric reflector paired with a second posterior plus powered aspheric reflector, each which respectively produce the stated posterior and negative and anterior and positive reflections, provides a single element optical system for a diagnostic and therapeutic gonioscopy lens that may be simply manufactured by means of diamond turning methods or with casting or molding procedures as are known in the art.

Referring to FIG. 5b, there is shown a ray tracing and schematic cross-sectional view of a lens layout of a second single element gonioscopy lens 90a according to the fourth embodiment of the invention. The exemplary lens of this figure has the same material composition and generally the same surface shape attributes as the lens shown in FIG. 5a and is different with respect to overall size and the magnification of the produced image. The description with respect to the light ray pathways of FIG. 5a applies to this lens. Referring to FIG. 5b, light rays of ray bundles 2e and 3e emanating from the stated iridocorneal and peripheral iris locations of anterior chamber 4e of eye 6e pass through the cornea 8e and tear layer of the eye and enter posterior contacting surface 94a of lens element 92a and continue to concave reflecting surface 96a from which each light ray is first reflected as a negative reflection in a posterior direction towards the axis of the lens, the light rays there forming an intermediate image within the lens. Each light ray proceeds in its respective direction across lens axis LA and continues to concave reflecting surface 98a from which it is next reflected as a positive reflection in an anterior direction, contributing to the formation of final and correctly oriented real image 100a. The light rays proceed from the real image in their respective directions towards surface 102a through which they are refracted and exit the lens.

Contacting surface 94a comprises a concave surface adapted for placement on the patient's cornea and has an apical radius of 7.7 mm and is aspheric. Reflecting surface 96a has an aspheric concave curvature with an apical radius of 17.16 mm and in combination with the displaced concave curvature of surface 102a comprises a lenticulated surface as the anterior surface of the lens. Reflecting surface 96a provides plus power, converging light rays directed to it from concave corneal contacting surface 94a. Reflecting surface 96a comprises an internally reflecting mirror-coated annular section having a 16 mm inner diameter that surrounds the outside diameter of anteriorly displaced refracting surface 102a. Reflecting surface 98a has an aspheric curvature with an apical radius of 10.65 mm and in combination with the displaced concave curvature of contacting surface 94a comprises a lenticulated surface at the posterior end of the lens. Reflecting surface 98a provides plus power, converging light rays directed to it from reflecting surface 96a. Reflecting surface 98a also comprises an internally reflecting mirror-coated annular section having a 6.0 mm inner diameter that surrounds the stemmed portion displacing it from concave contacting surface 94a. The reflective sections may be mirrored by means of vacuum deposition and protectively overcoated as previously described. Surface 102a has a polynomial defined aspheric curvature with both concave and convex attributes. Virtual image 104a is positioned internally within the lens approximately 4.5 mm from refracting surface 102a.

The exemplary lens as shown and described with reference to FIG. 5b, comprising a first anterior plus powered aspheric reflector paired with a second posterior plus powered aspheric reflector, each which respectively produce the stated posterior and negative and anterior and positive reflections, provides a single element optical system for a diagnostic and therapeutic gonioscopy lens that may be simply manufactured, is small in size and may be easily manipulated within the orbital area of the patient's eye. The single element lens of FIG. 5b may alternatively be made as a doublet lens optically coupled approximately along dotted line 98b. By so producing the lens in two portions, the anterior portion incorporating posterior reflecting surface 98a may be mirror coated prior to optically coupling to the posterior contacting portion incorporating surface 94a, thereby avoiding possible problems in mirror coating that may otherwise occur from shadowing caused by the peripheral flange portion of contacting surface 94a. As an alternative to polymethylmethacrylate as the material composition of the contacting portion incorporating surface 94a, the contacting portion may be composed of S-LAH58 optical glass or S-TIH6 optical glass (Available from Ohara Corporation) having an index of refraction of approximately Nd=1.805 and an Abbe number of approximately V=25.43. The meniscus glass contacting element design provides a durable and more scratch resistant contacting surface than does the polymethylmethacrylate and also provides substantial light converging power that in concert with the other lens surfaces produces an image having excellent quality and clarity. The meniscus glass element of either glass type may have a spherical concave contacting surface 94a with a radius of 8.0 mm, an opposing spherical convex surface with a radius of 6.0 mm and a center thickness of 1.2 mm and be optically coupled to the anterior portion incorporating reflecting surface 98a by means above outlined.

Referring to FIG. 6, there is shown a ray tracing and schematic cross-sectional view of a lens layout of an exemplary doublet indirect ophthalmoscopy contact lens 110 according to a fifth embodiment of the invention, wherein lens 110 comprises an optically coupled lens including posterior contacting and reflecting element 112 and anterior cap element 114. The lens receives light rays from points in the peripheral fundus and through refraction and reflection means similar to that of prior embodiments focuses the rays to form a real image as a continuous and interrupted annular section anterior of the examined eye. In this embodiment both the anterior and posterior reflecting surfaces comprise aspheric curvatures. Posterior contacting and reflecting element 112 is made of polymethylmethacrylate and anterior cap element 114 is made of optical quality polycarbonate having an index of refraction of approximately nd=1.585 and an Abbe number of approximately Vd=29.9 The two elements 112 and 114 are optically coupled at their interface using a suitable coupling material as previously described.

Referring to FIG. 6, light rays of ray bundles 116, 118, 120, 122, 124, 126, 128 and 130 emanating from equatorial-to-peripheral retinal sections of eye 132 pass through the vitreous humor 134, crystalline lens 136, anterior chamber 138, cornea 140 and tear layer of the eye and enter posterior contacting element 112 of lens 110 through corneal contacting surface 142 and continue to concave reflecting surface 144 from which each light ray is first reflected as a negative reflection in a posterior direction towards the axis of the lens, the light rays there forming an intermediate image within the lens. Each light ray proceeds in its respective direction across lens axis LA to concave reflecting surface 146 from which it is next reflected as a positive reflection in an anterior direction, contributing to the formation of final and correctly oriented real image 148. The light rays proceed from the real image in their respective directions towards interface 150, comprised of the anterior and posterior surfaces of lens elements 112 and 114 respectively, optically coupled with an interface material, and enter anterior cap element 114 and continue to surface 152 where they are refracted and exit the lens. The rays proceed towards biomicroscope objective lens aperture 154 and enter left and right microscope lenses 156 and 158, respectively, of the observing stereomicroscope. The stereomicroscope is adjusted to focus at virtual image 159 to provide an upright and correctly oriented view of the observed fundus structures of the eye.

In a manner similar to the prior exemplary gonioscopy lens embodiments light rays 116 to 130 emanating from the fundus of eye 132 span an area at the plane of biomicroscope objective lens 154 that exceeds the extent of the biomicroscope aperture and the left and right microscope lenses 156 and 158, thus insuring binocular and stereoscopic biomicroscope visualization of the observed image both when the biomicroscope is coaxial with the lens as shown and when the biomicroscope is moved off axis to bring peripheral image points to a more central location of the visual field of the biomicroscope.

Contacting surface 142 comprises a concave surface adapted for placement on the patient's cornea and has an apical radius of 6.5 mm and is aspheric. Optical interface 150 is the interface of the central refracting portions of the anterior and posterior surfaces respectively of lens elements 112 and 114. The curvature of interface 150 with respect to lens element 112 is aspheric and convex with an apical radius of 19.5 mm. Reflecting surface 144 is a continuation of the curvature comprising interface 150 and in combination with the curvature of interface 150 forms a continuous curvature as the anterior surface of lens element 112. Reflecting surface 144 provides plus power, converging light rays directed to it from concave corneal contacting surface 142. Reflecting surface 144 comprises an internally reflecting mirror-coated annular section having an 18 mm inner diameter that surrounds optical interface 150. Reflecting surface 146 has an aspheric curvature with an apical radius of 14.5 mm and in combination with the concave curvature of contacting surface 142 comprises a lenticulated surface as the posterior surface of lens element 112. Reflecting surface 146 provides plus power, converging light rays directed to it from reflecting surface 144. Reflecting surface 146 also comprises an internally reflecting mirror-coated annular section having a 9.8 mm inner diameter that surrounds concave contacting surface 142. The reflective sections may be mirrored by means of vacuum deposition and protectively overcoated as previously described. Surface 152 of lens element 114 has a polynomial defined aspheric curvature with concave attributes. Virtual image 159 is positioned internally within the lens approximately 2.5 mm from concave refracting surface 152 of anterior cap element 114.

The exemplary lens as shown and described with reference to FIG. 6, comprising a first anterior plus powered aspheric reflector paired with a second posterior plus powered aspheric reflector, each which respectively produce the stated posterior and negative and anterior and positive reflections, provides a correctly oriented wide field of view of the mid to peripheral fundus of the eye.

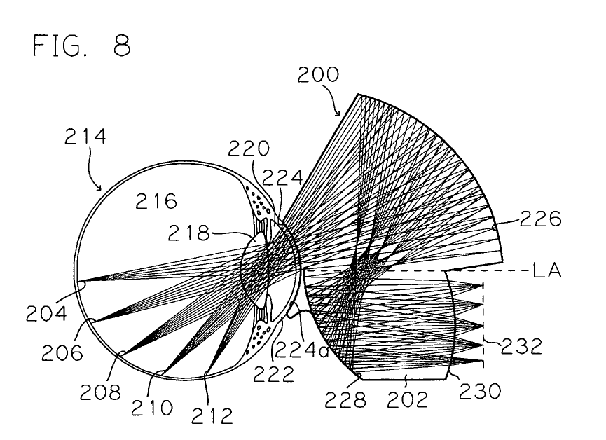

Referring to FIG. 7, there is shown a ray tracing and schematic cross-sectional view of a lens layout of an exemplary single element indirect ophthalmoscopy contact lens 160 according to a sixth embodiment of the invention. In this embodiment both the anterior and posterior reflecting surfaces comprise aspheric curvatures. Single lens element 162 is made of optical quality polymethylmethacrylate.

Referring to FIG. 7, light rays of ray bundles 164, 166, 168, 170, 172, 174, 176 and 178 emanating from equatorial-to-peripheral retinal sections of eye 180 pass through the vitreous humor 182, crystalline lens 184, anterior chamber 186, cornea 188 and tear layer of the eye and enter posterior contacting surface 190 of lens element 162 and continue to concave reflecting surface 192 from which each light ray is first reflected as a negative reflection in a posterior direction towards the axis of the lens, the light rays there forming an intermediate image within the lens. Each light ray proceeds in its respective direction across lens axis LA to concave reflecting surface 194 from which it is next reflected as a positive reflection in an anterior direction, contributing to the formation of final and correctly oriented real image 196. The light rays proceed from the real image in their respective directions towards surface 198 through which are refracted and exit the lens. The stereomicroscope is adjusted to focus at virtual image 199 to provide an upright and correctly oriented view of the observed structures of the eye.

Contacting surface 190 comprises a concave surface adapted for placement on the patient's cornea and has an apical radius of 6.5 mm and is aspheric. Reflecting surface 192 has an aspheric concave curvature with an apical radius of 19.5 mm and in combination with the concave curvature of surface 198 comprises a lenticulated surface as the anterior surface of the lens. Reflecting surface 192 provides plus power, converging light rays directed to it from concave corneal contacting surface 190. Reflecting surface 192 comprises an internally reflecting mirror-coated annular section having a 20 mm inner diameter that surrounds refracting surface 198. Reflecting surface 194 has an aspheric curvature with an apical radius of 14.5 mm and in combination with the concave curvature of contacting surface 190 comprises a lenticulated surface as the posterior surface of the lens. Reflecting surface 194 provides plus power, converging light rays directed to it from reflecting surface 192. Reflecting surface 194 also comprises an internally reflecting mirror-coated annular section having a 9.8 mm inner diameter that surrounds the concave contacting surface 190. The reflective sections may be mirrored by means of vacuum deposition and protectively overcoated as previously described. Surface 198 has a concave aspheric curvature with an apical radius of 20 mm. Virtual image 199 is located posterior of refracting surface 198.

The exemplary lens as shown and described with reference to FIG. 7, comprising a first anterior plus powered aspheric reflector paired with a second posterior plus powered aspheric reflector, each which respectively produce the stated posterior and negative and anterior and positive reflections, provides a correctly oriented wide field of view of the mid to peripheral fundus of the eye that may be simply manufactured as a single element optical system.

Referring to FIG. 8, there is shown a ray tracing and schematic cross-sectional view of an exemplary single element indirect ophthalmoscopy contact lens 200 according to a seventh embodiment of the invention. The lens of this embodiment provides a broad sectional field of view extending from the peripheral fundus to the central fundus region. The lens comprises non-annular anterior and posterior mirrored sections producing an upright real image offset from the optical centerline of the lens. Single lens element 202 is made of optical quality polymethylmethacrylate.

Referring to FIG. 8, light rays of ray bundles 204, 206, 208, 210, and 212 emanating from a sectional equatorial-to-peripheral retinal region of eye 214 passes through the vitreous humor 216, crystalline lens 218, anterior chamber 220, cornea 222 and tear layer of the eye and enter posterior contacting surface portion 224 of lens element 202 and continue to sectional concave reflecting surface 226 from which each light ray is first reflected as a negative reflection in a posterior direction towards the axis or centerline of the lens, the light rays there forming an intermediate image within the lens. Each light ray proceeds in its respective direction across line LA to sectional concave reflecting surface 228 from which it is next reflected as a positive reflection in an anterior direction towards surface 230 through which the light rays exit the lens and form final and correctly oriented real image 232. The stereomicroscope is adjusted to focus at real image 232 to provide an upright and correctly oriented view of the observed structures of the eye. Lens 200 may be selectively rotated and angled on the patient's eye by the practitioner in order to provide a fundus view including a broad area of the central retina or different regions of the peripheral fundus.

Contacting surface portions 224 and 224a form a continuous surface and together function as the contacting surface of the lens. The body of contacting portion 224a comprises a section of polymethylmethacrylate the side and back portions of which are cemented to the central exterior portion of mirror surface 228 and contacting portion 224. The continuous curvature formed by contacting portions 224 and 224a comprises a concave surface adapted for placement on the patient's cornea and has an apical radius of 7.7 mm and is aspheric. Reflecting surface 226 has an aspheric concave curvature with an apical radius of 18.5 mm and in combination with the convex curvature of surface 230 comprises a stepped and lenticulated surface as the anterior surface of the lens. Reflecting surface 226 provides plus power, converging light rays directed to it from concave corneal contacting surface portion 224. Reflecting surface 226 comprises an internally reflecting mirror-coated section adjacent refracting surface 230. Reflecting surface 228 has an aspheric curvature with an apical radius of 13.0 mm and in combination with the displaced concave curvature of contacting portions 224 and 224a comprises a lenticulated surface at the posterior end of the lens. Reflecting surface 228 provides plus power, converging light rays directed to it from reflecting surface 226. Reflecting surface 228 also comprises an internally reflecting mirror-coated section. The reflective sections may be mirrored by means of vacuum deposition and protectively overcoated as previously described. Surface 230 has a convex aspheric curvature with an apical radius of 25.0 mm. Real image 232 is positioned approximately 3 mm anterior of surface 230.

The exemplary lens as shown and described with reference to FIG. 8, comprising a first anterior plus powered aspheric reflector paired with a second posterior plus powered aspheric, each which respectively produce the stated posterior and negative and anterior and positive reflections, provides an expansive and correctly oriented sectional view of the mid to peripheral fundus of the eye that may be simply manufactured as a single element optical system.

The invention has been described in detail with respect to various embodiments and it will now be apparent from the foregoing to those skilled in the art that changes and modifications may be made without departing from the invention in its broader aspects. For example, the embodiments describing lenses of the present disclosure made of particular glass or plastic materials may instead be made with other polymers or with other optical glass types having any refractive index and Abbe value. It should be further understood that materials such as high temperature polymers suitable for optical applications may be used as replacements for acrylic or polycarbonate in order to accommodate high temperature sterilization procedures. As a further modification, additional lens elements may be incorporated into any of the embodiment designs without departing from the scope of the invention. Furthermore, any of the embodiments may incorporate a transparent or light filtering glass or plastic protective cover, and any refracting surfaces may be coated with an anti-reflective coating to lessen glaring reflection. It should be further understood that surfaces of lens embodiments using spherical curvatures may instead use aspheric curvatures and visa versa and that a lens design may be specifically adapted for use based on the particular design of the biomicroscope or other instrument used to capture the light rays as well as the refractive status of the examined eye. Furthermore, those lenses of each embodiment that are transparent through their central areas may be used to provide a direct view as a virtual image of the eye fundus through a center portion of the lens. It should be further understood that lenses of any of the embodiments may be provided with an aperture stop to modify image quality and contrast or with a centrally positioned light stop anterior of the location where the light rays cross the axis of the lens to prevent visualization of the central retina or laser energy entering the posterior chamber. It should be further understood that the illumination source may be other than that of a standard full wavelength white light illumination source, for example, the illumination may comprise light of monochromatic wavelengths or may comprise a laser or scanning laser, and that an image capture system used in conjunction with the lens may utilize such monochromatic or laser or laser scanned light, as is known to those skilled in the art. The invention, therefore, as defined in the appended claims is intended to cover all such changes and modifications as fall within the true spirit of the invention.

What is claimed is:

1. An ophthalmoscopic contact lens for viewing or treating a structure within an eye, comprising:
a contacting surface adapted for placement on a cornea of an eye including an anterior chamber and a posterior chamber;
an anterior reflecting surface positioned anterior of the contacting surface; and
a posterior reflecting surface positioned posterior of the anterior reflecting surface;
wherein a light ray emanating from the structure within the eye, entering the lens through the contacting surface and contributing to the formation of a correctly oriented real image of the structure, is reflected within the lens in an ordered sequence of reflections first as a negative reflection in a posterior direction by the anterior reflecting surface and next as a positive reflection in an anterior direction by the posterior reflecting surface.

2. The ophthalmoscopic contact lens of claim 1, wherein the anterior reflecting surface is at least partially concave.

3. The ophthalmoscopic contact lens of claim 2, wherein the posterior reflecting surface is at least partially concave.

4. The ophthalmoscopic contact lens of claim 1, further comprising:
a first part that includes at least a portion of the anterior reflecting surface; and
a second part that includes at least a portion of the posterior reflecting surface;
wherein the first reflection in the ordered sequence of reflections occurs in the first part and the next reflection in the ordered sequence of reflections occurs in the second part.

5. The ophthalmoscopic contact lens of claim 1, further comprising:
a first part that includes at least a portion of the posterior reflecting surface; and
a second part that includes at least a portion of the anterior reflecting surface;
wherein the first reflection in the ordered sequence of reflections occurs in the first part and the next reflection in the ordered sequence of reflections occurs in the second part.

6. The ophthalmoscopic contact lens of claim 1, further comprising a refracting surface positioned anterior to the posterior reflecting surface.

7. The ophthalmoscopic contact lens of claim 6, wherein the correctly oriented real image of the structure is formed posterior to the refracting surface.

8. The ophthalmoscopic contact lens of claim 6, wherein the correctly oriented real image of the structure is formed anterior to the refracting surface.

9. The ophthalmoscopic contact lens of claim 1, wherein the structure is within the anterior chamber of the eye.

10. The ophthalmoscopic contact lens of claim 9, wherein the lens is a singlet lens.

11. The ophthalmoscopic contact lens of claim 10, further comprising a refracting surface positioned anterior to the posterior reflecting surface;
wherein at least a portion of the refracting surface is a surface selected from the group consisting of a concave surface, a convex surface, a plano surface, and a polynomial surface having at least one concave portion and at least one convex portion.

12. The ophthalmoscopic contact lens of claim 11, wherein the anterior reflecting surface and the refracting surface together comprise a continuous surface.

13. The ophthalmoscopic contact lens of claim 11, wherein the posterior reflecting surface is spaced apart from the contacting surface in an anterior direction.

14. The ophthalmoscopic contact lens of claim 11, wherein the refracting surface is spaced apart from the anterior reflecting surface in an anterior direction.

15. The ophthalmoscopic contact lens of claim 9, wherein the lens is a doublet lens including a posterior portion and an anterior portion.

16. The ophthalmoscopic contact lens of claim 15, wherein the posterior portion and the anterior portion are optically coupled with an interface material.

17. The ophthalmoscopic contact lens of claim 16, wherein the interface material is selected from the group consisting of a liquid interface material, a gel interface material, and an optical cement interface material.

18. The ophthalmoscopic contact lens of claim 16, further comprising a refracting surface positioned anterior to the posterior reflecting surface, an anterior refracting surface, and a posterior refracting surface;
wherein the contacting surface, the posterior reflecting surface, the anterior reflecting surface, and the refracting surface together comprise the posterior portion of the lens;
further wherein the posterior refracting surface and the anterior refracting surface together comprise the anterior portion of the lens, and the interface material is positioned between the refracting surface and the posterior refracting surface.

19. The ophthalmoscopic contact lens of claim 18, wherein at least a portion of the anterior refracting surface is a surface selected from the group consisting of a concave surface, a convex surface, a plano surface, and a polynomial surface having at least one concave portion and at least one convex portion.

20. The ophthalmoscopic contact lens of claim 18, wherein at least a portion of the posterior refracting surface is a surface selected from the group consisting of a concave surface, a convex surface, a piano surface, and a polynomial surface having at least one concave portion and at least one convex portion.

21. The ophthalmoscopic contact lens of claim 18, wherein at least a portion of the refracting surface is a surface selected from the group consisting of a concave surface, a convex surface, a plano surface, and a polynomial surface having at least one concave portion and at least one convex portion.

22. The ophthalmoscopic contact lens of claim 18, wherein the refracting surface and the anterior reflecting surface together comprise a surface of continuous curvature.

23. The ophthalmoscopic contact lens of claim 16, further comprising a refracting surface positioned anterior to the posterior reflecting surface, an anterior refracting surface, and a posterior refracting surface;
wherein the contacting surface, the posterior reflecting surface, and the refracting surface together comprise the posterior portion of the lens;
further wherein the posterior refracting surface, the anterior reflecting surface, and the anterior refracting surface together comprise the anterior portion of the lens, and the interface material is positioned between the refracting surface and both the anterior reflecting surface and the posterior refracting surface.

24. The ophthalmoscopic contact lens of claim 16, further comprising a refracting surface positioned anterior to the posterior reflecting surface, an opposing refracting surface, and a posterior refracting surface;
wherein the contacting surface and the opposing refracting surface together comprise the posterior portion of the lens;
further wherein the posterior refracting surface, the posterior reflecting surface, the anterior reflecting surface, and the refracting surface together comprise the anterior portion of the lens, and the interface material is positioned between the opposing refracting surface and the posterior refracting surface.

25. The ophthalmoscopic contact lens of claim 24, wherein at least a portion of the opposing refractive surface is a surface selected from the group consisting of a concave surface, a convex surface, a piano surface, and a polynomial surface having at least one concave portion and at least one convex portion.

26. The ophthalmoscopic contact lens of claim 24, wherein at least a portion of the posterior refracting surface is a surface selected from the group consisting of a concave surface, a convex surface, a plano surface, and a polynomial surface having at least one concave portion and at least one convex portion.

27. The ophthalmoscopic contact lens of claim 24, wherein at least a portion of the refracting surface is a surface selected from the group consisting of a concave surface, a convex surface, a plano surface, and a polynomial surface having at least one concave portion and at least one convex portion.

28. The ophthalmoscopic contact lens of claim 24, wherein the refracting surface and the anterior reflecting surface together comprise a surface of continuous curvature.

29. The ophthalmoscopic contact lens of claim 24, wherein the posterior refracting surface and the posterior reflecting surface together comprise a surface of continuous curvature.

30. The ophthalmoscopic contact lens of claim 24, wherein the refracting surface and the anterior reflecting surface together comprise a first surface of continuous curvature and the posterior refracting surface and the posterior reflecting surface together comprise a second surface of continuous curvature.

31. The ophthalmoscopic contact lens of claim 16, further comprising a refracting surface positioned anterior to the posterior reflecting surface, an opposing refracting surface, and a posterior refracting surface;
wherein the contacting surface, the opposing refracting surface, and the posterior reflecting surface together comprise the posterior portion of the lens;
further wherein the posterior refracting surface, the anterior reflecting surface, and the refracting surface together comprise the anterior portion of the lens, and the interface material is positioned between the posterior refracting surface and both the posterior reflecting surface and the opposing refracting surface.

32. The ophthalmoscopic contact lens of claim 9, wherein the lens is a triplet lens including a posterior portion, an intermediate portion, and an anterior portion.

33. The ophthalmoscopic contact lens of claim 32, wherein the posterior portion and the intermediate portion are optically coupled with a first interface material, and the intermediate portion and the anterior portion are optically coupled with a second interface material.

34. The ophthalmoscopic contact lens of claim 33, wherein the first interface material is selected from the group consisting of a liquid interface material, a gel interface material, and an optical cement interface material, and the second interface material is selected from the group consisting of a liquid interface material, a gel interface material, and an optical cement interface material.

35. The ophthalmoscopic contact lens of claim 33, further comprising an opposing refracting surface, a first posterior refracting surface, a second posterior refracting surface, a first anterior refracting surface, and a second anterior refracting surface;
wherein the contacting surface and the opposing refracting surface together comprise the posterior portion of the lens;
wherein the anterior reflecting surface, the posterior reflecting surface, the first posterior refracting surface, and the first anterior refracting surface together comprise the intermediate portion of the lens;
wherein the second posterior refracting surface and the second anterior refracting surface together comprise the anterior portion of the lens;
further wherein the first interface material is positioned between the opposing refracting surface and the first posterior refracting surface, and the second interface material is positioned between the first anterior refracting surface and the second posterior refracting surface.

36. The ophthalmoscopic contact lens of claim 35, wherein at least a portion of the opposing refractive surface is a surface selected from the group consisting of a concave surface, a convex surface, a piano surface, and a polynomial surface having at least one concave portion and at least one convex portion.

37. The ophthalmoscopic contact lens of claim 35, wherein at least a portion of the first posterior refracting surface is a surface selected from the group consisting of a concave surface, a convex surface, a plano surface, and a polynomial surface having at least one concave portion and at least one convex portion.

38. The ophthalmoscopic contact lens of claim 35, wherein at least a portion of the second posterior refracting surface is a surface selected from the group consisting of a concave surface, a convex surface, a piano surface, and a polynomial surface having at least one concave portion and at least one convex portion.

39. The ophthalmoscopic contact lens of claim 35, wherein at least a portion of the first anterior refracting surface is a surface selected from the group consisting of a concave surface, a convex surface, a piano surface, and a polynomial surface having at least one concave portion and at least one convex portion.

40. The ophthalmoscopic contact lens of claim 35, wherein at least a portion of the second anterior refracting surface is a surface selected from the group consisting of a concave surface, a convex surface, a plano surface, and a polynomial surface having at least one concave portion and at least one convex portion.

41. The ophthalmoscopic contact lens of claim 35, wherein the first anterior refracting surface and the anterior reflecting surface together comprise a surface of continuous curvature.

42. The ophthalmoscopic contact lens of claim 35, wherein the first posterior refracting surface and the posterior reflecting surface together comprise a surface of continuous curvature.

43. The ophthalmoscopic contact lens of claim 35, wherein the first anterior refracting surface and the anterior reflecting surface together comprise a surface of continuous curvature, and the first posterior refracting surface and the posterior reflecting surface together comprise a surface of continuous curvature.

44. The ophthalmoscopic contact lens of claim 33, further comprising an opposing refracting surface, a first posterior refracting surface, a second posterior refracting surface, a first anterior refracting surface, and a second anterior refracting surface;
wherein the contacting surface, the opposing refracting surface, and the posterior reflecting surface together comprise the posterior portion of the lens;
wherein the first posterior refracting surface and the first anterior refracting surface together comprise the intermediate portion of the lens;
wherein the second posterior refracting surface, the anterior reflecting surface, and the second anterior refracting surface together comprise the anterior portion of the lens;
further wherein the first interface material is positioned between the first posterior refracting surface and both the posterior reflecting surface and the opposing refracting surface, and the second interface material is positioned between the first anterior refracting surface and both the anterior reflecting surface and second posterior refracting surface.

45. The ophthalmoscopic contact lens of claim 33, further comprising an opposing refracting surface, a first posterior refracting surface, a second posterior refracting surface, a first anterior refracting surface, and a second anterior refracting surface;
wherein the contacting surface, the opposing refracting surface, and the posterior reflecting surface together comprise the posterior portion of the lens;
wherein the first posterior refracting surface, the anterior reflecting surface, and the first anterior refracting surface together comprise the intermediate portion of the lens;
wherein the second posterior refracting surface and the second anterior refracting surface together comprise the anterior portion of the lens;
further wherein the first interface material is positioned between the first posterior refracting surface and both the posterior reflecting surface and the opposing refracting surface, and the second interface material is positioned between the first anterior refracting surface and the second posterior refracting surface.

46. The ophthalmoscopic contact lens of claim 33, further comprising an opposing refracting surface, a first posterior refracting surface, a second posterior refracting surface, a first anterior refracting surface, and a second anterior refracting surface;
wherein the contacting surface and the opposing refracting surface together comprise the posterior portion of the lens;
wherein the first posterior refracting surface, the posterior reflecting surface, and the first anterior refracting surface together comprise the intermediate portion of the lens;
wherein the second posterior refracting surface, the anterior reflecting surface, and the second anterior refracting surface together comprise the anterior portion of the lens;
further wherein the first interface material is positioned between the opposing refracting surface and the first posterior refracting surface, and the second interface material is positioned between the first anterior refracting surface and both the anterior reflecting surface and the second posterior refracting surface.

47. The ophthalmoscopic contact lens of claim 1, wherein the structure is within the posterior chamber of the eye.

48. The ophthalmoscopic contact lens of claim 47, wherein the lens is a singlet lens.

49. The ophthalmoscopic contact lens of claim 48, further comprising a refracting surface positioned anterior to the posterior reflecting surface;
wherein the refracting surface is a surface selected from the group consisting of a concave surface, a convex surface, a plano surface, and a polynomial surface having at least one concave portion and at least one convex portion.

50. The ophthalmoscopic contact lens of claim 47, wherein the lens is a doublet lens including a posterior portion and an anterior portion.

51. The ophthalmoscopic contact lens of claim 50, wherein the posterior portion and the anterior portion are optically coupled with an interface material.

52. The ophthalmoscopic contact lens of claim 51, wherein the interface material is selected from the group consisting of a liquid interface material, a gel interface material and, an optical cement interface material.

53. The ophthalmoscopic contact lens of claim 51, further comprising, a refracting surface positioned anterior to the posterior reflecting surface, an anterior refracting surface, and a posterior refracting surface;
wherein the contacting surface, the posterior reflecting surface, the anterior reflecting surface, and the refracting surface together comprise the posterior portion of the lens;
further wherein the posterior refracting surface and the anterior refracting surface together comprise the anterior portion of the lens, and the interface material is positioned between the refracting surface and the posterior refracting surface.

54. The ophthalmoscopic contact lens of claim 51, further comprising a refracting surface positioned anterior to the posterior reflecting surface, an anterior refracting surface and a posterior refracting surface;
wherein the contacting surface, the posterior reflecting surface, and the refracting surface together comprise the posterior portion of the lens;
further wherein the posterior refracting surface, the anterior reflecting surface, and the anterior refracting surface together comprise the anterior portion of the lens, and the interface material is positioned between the refracting surface and both the anterior reflecting surface and the posterior refracting surface.

55. The ophthalmoscopic contact lens of claim 51, further comprising a refracting surface positioned anterior to the posterior reflecting surface, opposing refracting surface, and a posterior refracting surface;
wherein the contacting surface and the opposing refracting surface together comprise the posterior portion of the lens;
further wherein the posterior refracting surface, the posterior reflecting surface, the anterior reflecting surface, and the refracting surface together comprise the anterior portion of the lens, and the interface material is positioned between the opposing refracting surface and the posterior refracting surface.

56. The ophthalmoscopic contact lens of claim 51, further comprising a refractive surface positioned anterior to the posterior reflecting surface, an opposing refracting surface, and a posterior refracting surface;
wherein the contacting surface, the opposing refracting surface, and the posterior reflecting surface together comprise the posterior portion of the lens;
further wherein the posterior refracting surface, the anterior reflecting surface, and the refracting surface together comprise the anterior portion of the lens, and the interface material is positioned between the posterior refracting surface and both the posterior reflecting surface and the opposing refracting surface.

57. The ophthalmoscopic contact lens of claim 47, wherein the lens is a triplet lens including a posterior portion, an intermediate portion, and an anterior portion.

58. The ophthalmoscopic contact lens of claim 57, wherein the posterior portion and the intermediate portion are optically coupled with a first interface material, and the intermediate portion and the anterior portion are optically coupled with a second interface material.

59. The ophthalmoscopic contact lens of claim 58, wherein the first interface material is selected from the group consisting of a liquid interface material, a gel interface material, and an optical cement interface material, and the second interface material is selected from the group consisting of a liquid interface material, a gel interface material, and an optical cement interface material.

60. The ophthalmoscopic contact lens of claim 58, further comprising an opposing refracting surface, a first posterior refracting surface, a second posterior refracting surface, a first anterior refracting surface, and a second anterior refracting surface;
wherein the contacting surface and the opposing refracting surface together comprise the posterior portion of the lens;
wherein the anterior reflecting surface, the posterior reflecting surface, the first posterior refracting surface, and the first anterior refracting surface together comprise the intermediate portion of the lens;
wherein the second posterior refracting surface and the second anterior refracting surface together comprise the anterior portion of the lens;
further wherein the first interface material is positioned between the opposing refracting surface and the first posterior refracting surface, and the second interface material is positioned between the first anterior refracting surface and the second posterior refracting surface.

61. The ophthalmoscopic contact lens of claim 58, further comprising an opposing refracting surface, a first posterior refracting surface, a second posterior refracting surface, a first anterior refracting surface, and a second anterior refracting surface;
wherein the contacting surface, the opposing refracting surface, and the posterior reflecting surface together comprise the posterior portion of the lens;
wherein the first posterior refracting surface and the first anterior refracting surface together comprise the intermediate portion of the lens;
wherein the second posterior refracting surface, the anterior reflecting surface, and the second anterior refracting surface together comprise the anterior portion of the lens;
further wherein the first interface material is positioned between the first posterior refracting surface and both the posterior reflecting surface and the opposing refracting surface, and the second interface material is positioned between the first anterior refracting surface and both the anterior reflecting surface and the second posterior refracting surface.

62. The ophthalmoscopic contact lens of claim 58, further comprising an opposing refracting surface, a first posterior refracting surface, a second posterior refracting surface, a first anterior refracting surface, and a second anterior refracting surface;
wherein the contacting surface, the opposing refracting surface, and the posterior reflecting surface together comprise the posterior portion of the lens;
wherein the first posterior refracting surface, the anterior reflecting surface, and the first anterior refracting surface together comprise the intermediate portion of the lens;
wherein the second posterior refracting surface and the second anterior refracting surface together comprise the anterior portion of the lens;
further wherein the first interface material is positioned between the first posterior refracting surface and both the posterior reflecting surface and the opposing refracting surface, and the second interface material is positioned between the first anterior refracting surface and the second posterior refracting surface.

63. The ophthalmoscopic contact lens of claim 58, further comprising an opposing refracting surface, a first posterior refracting surface, a second posterior refracting surface, a first anterior refracting surface, and a second anterior refracting surface;
wherein the contacting surface and the opposing refracting surface together comprise the posterior portion of the lens;
wherein the first posterior refracting surface, the posterior reflecting surface, and the first anterior refracting surface together comprise the intermediate portion of the lens;
wherein the second posterior refracting surface, the anterior reflecting surface, and the second anterior refracting surface together comprise the anterior portion of the lens;
further wherein the first interface material is positioned between the opposing refracting surface and the first posterior refracting surface, and the second interface material is positioned between the first anterior refracting surface and both the anterior reflecting surface and the second posterior refracting surface.

64. The ophthalmoscopic contact lens of claim 1, further comprising an image sensor for converting light contributing to the formation of the correctly oriented real image to an electrical signal.

65. The ophthalmoscopic contact lens of claim 64, wherein the correctly oriented real image is positioned on the image sensor.

66. The ophthalmoscopic contact lens of claim 65, wherein the image sensor converts at least a portion of the light contributing to the correctly oriented real image to the electrical signal.

67. The ophthalmoscopic contact lens of claim 66, further comprising an analog-to-digital converter and a computer readable medium.

68. The ophthalmoscopic contact lens of claim 67, wherein the analog-to-digital converter digitizes the electrical signal for storage on the computer readable medium.

69. The ophthalmoscopic contact lens of claim 67, wherein the analog-to-digital converter digitizes the electrical signal for display on a displaying device.

70. The ophthalmoscopic contact lens of claim 65, wherein the image sensor is a charged-coupled device.

71. The ophthalmoscopic contact lens of claim 65, wherein the image sensor is a complementary metal-oxide-semiconductor active-pixel sensor.

72. The ophthalmoscopic contact lens of claim 64, wherein the image sensor is positioned within a camera adapted to capture the correctly oriented real image.

73. A ophthalmoscopic contact lens camera for capturing an image of a structure within an eye, comprising:
a contact lens including:
a contacting surface adapted for placement on the cornea of an eye including an anterior chamber and a posterior chamber;
an anterior reflecting surface positioned anterior to the contacting surface; and
a posterior reflecting surface positioned posterior of the anterior reflecting surface;
a camera body; and
a light sensitive surface adapted to capture an image;
wherein a light ray emanating from the structure within the eye and contributing to the formation of a correctly oriented real image of the structure is reflected within the lens in an ordered sequence of reflections first as a negative reflection in a posterior direction by the anterior reflecting surface and next as a positive reflection in an anterior direction by the posterior reflecting surface;
further wherein the camera is adapted to position the light sensitive surface at a plane of the correctly oriented real image to capture the image of the structure within the eye.

74. A method for manufacturing an ophthalmoscopic contact lens:
forming a contacting surface adapted for placement on a cornea of an eye including an anterior chamber and a posterior chamber and further adapted to permit entrance into the lens of a light ray emanating from a structure within the eye and contributing to the formation of a correctly oriented real image of the structure;
forming an anterior reflecting surface positioned anterior of the contacting surface and adapted to reflect the light ray in a posterior direction as a negative reflection that is a first reflection in an ordered sequence of reflections; and
forming a posterior reflecting surface positioned posterior of the anterior reflecting surface and adapted to reflect the light ray in an anterior direction as a positive reflection that is a next reflection in the ordered sequence of reflections.

75. The method of claim 74, further comprising forming a refracting surface positioned anterior to the posterior reflecting surface.

76. The method of claim 75, wherein the contacting surface, the anterior reflecting surface, the posterior reflection surface, and the refracting surface are adapted to form the correctly oriented real image posterior to the refracting surface.

77. The method of claim 75, wherein the contacting surface, the anterior reflecting surface, the posterior reflection surface, and the refracting surface are adapted to form the correctly oriented real image anterior to the refracting surface.

78. The method of claim 74, wherein the contact surface, the posterior reflecting surface, and the anterior reflecting surface are formed as a singlet lens.

79. The method of claim 74, further comprising:
forming a refracting surface positioned anterior to the posterior reflecting surface;
forming an anterior refracting surface; and
forming a posterior refracting surface.

80. The method of claim 79, wherein the contacting surface, the posterior reflecting surface, the anterior reflecting surface, and the refracting surface are formed as a posterior portion of a doublet lens, and the anterior refracting surface and the posterior refracting surface are formed as an anterior portion of the doublet lens;
further wherein an interface material is positioned between the refracting surface and the posterior refracting surface to optically couple the anterior portion and the posterior portion.

81. The method of claim 80, further comprising selecting the interface material from the group consisting of a liquid interface material, a gel interface material, and an optical cement interface material.

82. The method of claim 79, wherein the contacting surface, the posterior reflecting surface, and the refracting surface are formed as a posterior portion of a doublet lens, and the posterior refracting surface, the anterior reflecting surface, and the anterior refracting surface are formed as an anterior portion of the doublet lens;
further wherein an interface material is positioned between the refracting surface and both the anterior reflecting surface and the posterior refracting surface to optically couple the anterior portion and the posterior portion.

83. The method of claim 79, further comprising forming an opposing refracting surface;
wherein the contacting surface and the opposing refracting surface are formed as a posterior portion of a doublet lens, and the posterior refracting surface, the posterior reflecting surface, the anterior reflecting surface, and the refracting surface are formed as an anterior portion of the doublet lens;
further wherein an interface material is positioned between the opposing refracting surface and the posterior refracting surface to optically couple the anterior portion and the posterior portion.

84. The method of claim 79, further comprising forming an opposing refracting surface;
wherein the contacting surface, the opposing refracting surface, and the posterior reflecting surface are formed as a posterior portion of a doublet lens, and the posterior refracting surface, the anterior reflecting surface, and the refracting surface are formed as an anterior portion of the doublet lens;
further wherein an interface material is positioned between the posterior refracting surface and both the posterior reflecting surface and the opposing refracting surface to optically couple the anterior portion and the posterior portion.

85. The method of claim 74, further comprising:
forming an opposing refracting surface;
forming a first anterior refracting surface;
forming a first posterior refracting surface;
forming a second anterior refracting surface; and
forming a second posterior refracting surface.

86. The method of claim 88, wherein the contacting surface and the opposing refracting surface are formed as a posterior portion of a triplet lens;
wherein the anterior reflecting surface, the posterior reflecting surface, the first posterior refracting surface, and the first anterior refracting surface are formed as an intermediate portion of the triplet lens;
wherein the second posterior refracting surface and the second anterior refracting surface are formed as an anterior portion of the triplet lens;
further wherein a first interface material is positioned between the opposing refracting surface and the first posterior refracting surface to optically couple the posterior portion and the intermediate portion, and a second interface material is positioned between the first anterior refracting surface and the second posterior refracting surface to optically couple the intermediate portion and the anterior portion.

87. The method of claim 86, further comprising:
selecting the first interface material from the group consisting of a liquid interface material, a gel interface material, and an optical cement interface material; and
selecting the second interface material from the group consisting of a liquid interface material, a gel interface material, and an optical cement interface material.

88. The method of claim 85, wherein the contacting surface, the opposing refracting surface, and the posterior reflecting surface are formed as a posterior portion of a triplet lens;
wherein the first posterior refracting surface and the first anterior refracting surface are formed as an intermediate portion of the triplet lens;
wherein the second posterior refracting surface, the anterior reflecting surface, and the second anterior refracting surface are formed as an anterior portion of the triplet lens;
further wherein a first interface material is positioned between the first posterior refracting surface and both the posterior reflecting surface and the opposing refracting surface to optically couple the posterior portion and the intermediate portion, and a second interface material is positioned between the first anterior refracting surface and both the anterior reflecting surface and the second posterior refracting surface to optically couple the intermediate portion and the anterior portion.

89. The method of claim 85, wherein the contacting surface, the opposing refracting surface, and the posterior reflecting surface are formed as a posterior portion of a triplet lens;
wherein the first posterior refracting surface, the anterior reflecting surface, and the first anterior refracting surface are formed as an intermediate portion of the triplet lens;
wherein the second posterior refracting surface and the second anterior refracting surface are formed as an anterior portion of the triplet lens;
further wherein a first interface material is positioned between the first posterior refracting surface and both the posterior reflecting surface and the opposing refracting surface to optically couple the posterior portion and the intermediate portion, and a second interface material is positioned between the first anterior refracting surface and the second posterior refracting surface to optically couple the intermediate portion and the anterior portion.

90. The method of claim 85, wherein the contacting surface and the opposing refracting surface are formed as a posterior portion of a triplet lens;
wherein the first posterior refracting surface, the posterior reflecting surface, and the first anterior refracting surface are formed as an intermediate portion of the triplet lens;
wherein the second posterior refracting surface, the anterior reflecting surface, and the second anterior refracting surface are formed as an anterior portion of the triplet lens;
further wherein a first interface material is positioned between the opposing refracting surface and the first posterior refracting surface to optically couple the posterior portion and the intermediate portion, and a second interface material is positioned between the first anterior refracting surface and both the anterior reflecting surface and the second posterior refracting surface to optically couple the intermediate portion and the anterior portion.

91. An ophthalmoscopic contact lens for viewing or treating a structure within an eye, comprising:
means for contacting a surface of a cornea of an eye including an anterior chamber and a posterior chamber, the means for contacting a surface of a cornea adapted to permit entrance into the lens of a light ray emanating from the structure within the eye and contributing to the formation of a correctly oriented real image of the structure;
anterior means for reflecting, positioned anterior of the means for contacting a surface of a cornea and adapted to reflect the light ray in a posterior direction as a negative reflection that is a first reflection in an ordered sequence of reflections; and
posterior means for reflecting, positioned posterior of the anterior means for reflecting and adapted to reflect the light ray in an anterior direction as a positive reflection that is a next reflection in the ordered sequence of reflections.

92. The ophthalmoscopic contact lens of claim 91, further comprising a means for refracting positioned anterior to the posterior means for reflecting.

93. The ophthalmoscopic contact lens of claim 92, wherein the correctly oriented real image of the structure is formed posterior to the means for refracting.

94. The ophthalmoscopic contact lens of claim 92, wherein the correctly oriented real image of the structure is formed anterior to the means for refracting.

95. The ophthalmoscopic contact lens of claim 91, wherein the contact lens is a singlet lens.

96. The ophthalmoscopic contact lens of claim 91, wherein the contact lens is a doublet lens including a posterior portion and an anterior portion.

97. The ophthalmoscopic contact lens of claim 96, wherein the posterior portion and anterior portion are optically coupled by a means for interfacing.

98. The ophthalmoscopic contact lens of claim 97, further comprising a means for refracting positioned anterior to the posterior means for reflecting, an anterior means for refracting, and a posterior means for refracting;
wherein the means for contacting, the posterior means for reflecting, the anterior means for reflecting, and the means for refracting comprise the posterior portion of the lens;
further wherein the posterior means for refracting and the anterior means for refracting comprise the anterior portion of the lens, and the means for interfacing is positioned between the means for refracting and the posterior means for refracting.

99. The ophthalmoscopic contact lens of claim 97, further comprising a means for refracting positioned anterior to the posterior means for reflecting, an anterior means for refracting, and a posterior means for refracting;
wherein the means for contacting, the posterior means for reflecting, and the means for refracting comprise the posterior portion of the lens;
further wherein the posterior means for refracting, the anterior means for reflecting, and the anterior means for refracting comprise the anterior portion of the lens, and the means for interfacing is positioned between the means for refracting and both the anterior means for reflecting and the posterior means for refracting.

100. The ophthalmoscopic contact lens of claim 98, further comprising a means for refracting positioned anterior to the posterior means for reflecting, an opposing means for refracting, and a posterior means for refracting;
wherein the means for contacting and the opposing means for refracting comprise the posterior portion of the lens;
further wherein the posterior means for refracting, the posterior means for reflecting, the anterior means for reflecting, and the means for refracting comprise the anterior portion of the lens, and the means for interfacing is positioned between the opposing means for refracting and the posterior means for refracting.

101. The ophthalmoscopic contact lens of claim 97, further comprising a means for refracting positioned anterior to the posterior means for reflecting, an opposing means for refracting, and a posterior means for refracting;
wherein the means for contacting, the opposing means for refracting, and the posterior means for reflecting comprise the posterior portion of the lens;
further wherein the posterior means for refracting, the anterior means for reflecting, and the means for refracting comprise the anterior portion of the lens, and the means for interfacing is positioned between the posterior means for refracting and both the posterior means for reflecting and the opposing means for refracting.

102. The ophthalmoscopic contact lens of claim 91, wherein the contact lens is a triplet lens including a posterior portion, an intermediate portion, and an anterior portion.

103. The ophthalmoscopic contact lens of claim 102, wherein the posterior portion and intermediate portion are optically coupled by a first means for interfacing, and the intermediate portion and anterior portion are optically coupled by a second means for interfacing.

104. The ophthalmoscopic contact lens of claim 103, further comprising an opposing means for refracting, a first posterior means for refracting, a second posterior means for refracting, a first anterior means for refracting, and a second anterior means for refracting;
wherein the means for contacting and the opposing means for refracting comprise the posterior portion of the lens;
wherein the anterior means for reflecting, the posterior means for reflecting, the first posterior means for refracting, and the first anterior means for refracting comprise the intermediate portion of the lens;
wherein the second posterior means for refracting and the second anterior means for refracting comprise the anterior portion of the lens;
further wherein the first means for interfacing is positioned between the opposing means for refracting and the first posterior means for refracting, and the second means for interfacing is positioned between the first anterior means for refracting and the second posterior means for refracting.

105. The ophthalmoscopic contact lens of claim 103, further comprising an opposing means for refracting, a first posterior means for refracting, a second posterior means for refracting, a first anterior means for refracting, and a second anterior means for refracting;
wherein the means for contacting, the opposing means for refracting, and the posterior means for reflecting comprise the posterior portion of the lens;
wherein the first posterior means for refracting and the first anterior means for refracting comprise the intermediate portion of the lens;
wherein the second posterior means for refracting, the anterior means for reflecting, and the second anterior means for refracting comprise the anterior portion of the lens;
further wherein the first means for interfacing is positioned between the first posterior means for refracting and both the posterior means for reflecting and the opposing means for refracting, and the second means for interfacing is positioned between the first means for anterior refracting and both the anterior means for reflecting and second posterior means for refracting.

106. The ophthalmoscopic contact lens of claim 103, further comprising an opposing means for refracting, a first posterior means for refracting, a second posterior means for refracting, a first anterior means for refracting, and a second anterior means for refracting;
wherein the means for contacting, the opposing means for refracting, and the posterior means for reflecting comprise the posterior portion of the lens;
wherein the first posterior means for refracting, the anterior means for reflecting, and the first anterior means for refracting comprise the intermediate portion of the lens;
wherein the second posterior means for refracting and the second anterior means for refracting comprise the anterior portion of the lens;
further wherein the first means for interfacing is positioned between the first posterior means for refracting and both the posterior means for reflecting and the opposing means for refracting, and the second means for interfacing is positioned between the first anterior means for refracting and the second posterior means for refracting.

107. The ophthalmoscopic contact lens of claim 103, further comprising an opposing means for refracting, a first posterior means for refracting, a second posterior means for refracting, a first anterior means for refracting, and a second anterior means for refracting;
wherein the means for contacting and the opposing means for refracting comprise the posterior portion of the lens;
wherein the first posterior means for refracting, the posterior means for reflecting, and the first anterior means for refracting comprise the intermediate portion of the lens;
wherein the second posterior means for refracting, the anterior means for reflecting, and the second anterior means for refracting comprise the anterior portion of the lens; further wherein the first means for interfacing is positioned between the opposing means for refracting and the first posterior means for refracting, and the second means for interfacing is positioned between the first anterior means for refracting and both the anterior means for reflecting and the second posterior means for refracting.
